(19) United States
(12) Patent Application Publication
(10) Pub. No.: US 2010/0291543 A1
(43) Pub. Date: Nov. 18, 2010

(54) HOMOGENEOUS IN VITRO FEC ASSAYS AND COMPONENTS

(76) Inventors: Rachel De Las Heras, Queensland (AU); Scott Robert Fry, Queensland (AU); Jennifer Ann McCourt, Queensland (AU); Jun Li, Queensland (AU); Ronald George Duggleby, Queensland (AU)

Correspondence Address: FOLEY HOAG, LLP (w/ISA);PATENT GROUP, 155 SEAPORT BLVD., BOSTON, MA, 02210-2600, US

(21) Appl. No.: 12/525,978

(22) PCT Filed: Apr. 19, 2007
(86) PCT No.: PCT/AU07/00508

§ 371 Date: May 24, 2010

(30) Foreign Application Priority Data
Feb. 5, 2007 (AU) 2007900530

(51) Int. Cl. C12Q 1/70; G01N 33/554

(52) U.S. Cl. 435/5; 435/7.32; 530/387.1

(57) ABSTRACT

Reporter fragments, reporter components, and systems adapted to detect analytes in homogeneous in vitro assays are provided, such assays employing these systems, and methods of making and using same. Particular embodiments include isolated and purified reporter fragments displaying enhanced solubility, reduced aggregation, resistance to inhibitors, and enhanced suitability for use in homogeneous in vitro assays.

### FIELD OF THE INVENTION

This invention relates generally to the field of molecular biology and diagnostics. More specifically, the invention provides reporter fragment systems and components specifically adapted to detect analytes in homogeneous in vitro assays, such assays employing these components, diagnostic systems, and methods of making and using same.

### BACKGROUND OF THE INVENTION

Assays based upon reporter fragment systems are known as Protein Fragment Complementation assays (PCAs), Forced Enzyme Complementation (FEC) assays or systems, or as interaction-dependent protein association systems. These assays are described generally in, for example: WO 01/71702; U.S. Pat. Nos. 6,270,964; 6,294,330; 6,428,951; 6,342,345, 6,828,099 and published US Patent Application 20030175836. As used herein, Forced Enzyme Complementation, or FEC, assays will refer generically to such assays.

The fundamental principle that underlies FEC is diagrammed in FIG. 1. These assays are typically characterised by the use of a protein fragment pair comprised of a first and second member that may functionally reassemble into a reporter protein having a directly detectable signal, such as a visible phenotypic change or antibiotic resistance. The key aspect of the assay's utility is that the functional reassembly of the reporter protein is dependent upon the interaction, i.e. binding or attraction, of moieties present within each member of the fragment pair that have been attached to the reporter protein fragments. As used herein, these interaction moieties are termed “interactors” or “interactor domains”. Interactor domains are separate moieties or domains contained within each member of the fragment pair constructs. Interactor domains may be joined directly to the reporter fragment domains or may be joined by a linker domain. The protein fragment pair members are typically constructed so that, absent the presence of interactor domains on each member of the pair, the reporter fragments do not spontaneously functionally reassemble. Thus, the interactor domains of each member of the pair assist the reconstitution of a functional reporter protein from its fragments.

Functional, in vivo FEC assays have been constructed using several different reporter proteins. See, for example, those disclosed in WO 01/71702; U.S. Pat. Nos. 6,270,964; 6,294,330; 6,428,951; 6,342,345, 6,828,099 and published US Patent Application 20030175836.

Class A Beta-lactamases (particularly TEM-1 Beta-lactamase) have been of particular interest in the development of FEC assays because they are monomeric, of relatively small size, and the crystal structure is known (WO 00/71702 and Jelsch et al., Proteins Struct. Funct. (1993) 16:364ff). Beta-lactamases may be expressed in either prokaryotic or eukaryotic systems. Examples of the use of Beta-lactamase in the design and development of FEC assays may be found in WO 01/71702; U.S. Pat. Nos. 6,270,964; 6,294,330; 6,428,951; 6,342,345, 6,828,099, published US Patent Applications 20030175836 and 20060094014 and Ooi, et al. (2006) Biochemistry 45:3620-3625.

Cantor et al. (Published US patent application 20060094014) suggest FEC for use in detecting target nucleic acid sequences wherein the interactor domains are nucleic acids. The interaction necessary for effective FEC assay performance results from the formation of nucleic acid complementation complexes (a result of typical Watson-Crick base pair sequence recognition) among the nucleic acids involved. Thus, in the methods of Cantor et al., the target analyte and the interactor domains of each FEC fragment are all nucleic acids. Each member of each fragment pair is thus a mixed construct of protein reporter fragment pairs derived from Beta-lactamase and nucleic acid interactor domains linked to those reporter fragments. Ooi et al. (2006) described a similar system using Beta-lactamase reporter fragment pairs. Stains et al. (2005) J. Am. Chem. Soc. 127: 10782-10783 and Stains et al. (2006) J. Am. Chem. Soc. 128: 9761-9765 described FEC based assays utilizing fragments of green fluorescent protein (GFP) as reporter fragment pairs and zinc-finger and methyl-CpG binding proteins as interactor domains. These systems are designed to detect sequence specific or methylated polynucleotide sequences through fluorescence of reconstituted GFP.

The creation of broadly applicable in vitro homogeneous assays based upon the principle of FEC necessarily presents novel challenges to the design and implementation of FEC. These challenges arise from the particular assay environment created within a homogeneous assay format, the nature and source of analytes intended for detection, and the manufacturing challenges associated with the production of such homogeneous assays.

Homogeneous assays are typically constituted of isolated, purified components in order to ensure specificity, reliability, manufacturing ease and robust characteristics in use. The design of appropriate protein fragment pairs (including appropriate reporter fragments, interactor domains, linking domains, and whole fusion constructs or reporter fragment pairs incorporating each) as well as robust assay conditions that also produce the necessary solubility, stability, and amenability to manufacture and ultimate diagnostic use is crucial to creating a broadly applicable homogeneous assay platform. In contrast to in vivo FEC assay conditions, in vitro diagnostic assay conditions are extra-cellular and relatively harsh and may not be conducive to appropriate protein folding and protein-protein interactions that may readily occur in vivo.

Additional challenges are presented by a homogeneous assay platform. Central to the development of the homogeneous assay platform is the elimination of wash steps. Diagnostic tests in a homogeneous assay platform therefore may be carried out in the presence of serum, or at least some serum components. Thus, sample fractions containing analytes may be processed without washing. The absence of washing is likely to leave homogeneous platforms, especially those based upon FEC, susceptible to cross-reactivity, interference and inhibitory effects of serum components or other contaminants.

For example, Beta-lactamases provide bacterial resistance to Beta-lactam antibiotics (Chaibi et al. 1999, J. Antimicrob. Chemother. 43(4): 447-58). Consequently, drugs designed for the treatment of bacterial infections, when present in patient serum, may interfere with assay performance. Indeed, Beta-lactam antibiotic formulations used for human administration (oral and intravenous) may include the use of irreversible inhibitors of Beta-lactamase (TEM-1) to increase the efficacy of antibiotics used. Commonly used example inhibitors include derivatives of penicillin, and penam or cepham derivatives. Examples of such inhibitors, their synthesis and use are described in U.S. Pat. Nos. 6,936,711; 6,900,184; 6,395,726; 6,207,661; 5,763,603; 5,686,441; 4,958,020; 4,933,444; 4,898,939; 4,895,941; 4,891,369; 4,668,514; 4,626,384; and 4,529,592, each of which is expressly incorporated by reference in their entirety. Common combinations of antibiotics and inhibitors may be known by their trade names or informal names. For example: Piperacillin and Tazobactam; Ampicillin sodium and Sulbactam sodium; and Amoxicillin and Clavulanic acid. Additional combinations and inhibitors are known in the art.

Homogeneous assays utilizing Beta-lactamase as a reporter enzyme will be susceptible to these Beta-lactamase inhibitors. For example, β-lactamase inhibitor serum Cₘₐₓ levels observed following antibiotic administration may be sufficiently high enough to inhibit β-lactamase. Therefore, in addition to the challenges of implementing FEC in an in vitro assay generally, there are additional challenges presented by implementation of FEC in an in vitro homogeneous assay platform.

### SUMMARY OF INVENTION

The invention provides modified reporter fragments (α and ω fragments) based upon TEM-1 Beta-lactamase that display desirable characteristics for use in in vitro homogeneous FEC assays, such as enhanced solubility, stability, sensitivity and/or resistance to enzyme inhibitors, e.g. inhibitors that may be present in samples which it is desired to test in the assays. These desirable characteristics displayed by the protein fragments of the invention include enhanced amenability to isolation and purification of the reporter fragments and the reporter fragment pairs for subsequent constitution of an operable in vitro homogeneous assay. The invention further specifically provides reporter protein fragments that display enhanced solubility and reduced aggregation under homogeneous in vitro assay conditions while maintaining or improving upon the sensitivity or specificity of the assay in homogeneous assay platform format and use.

Accordingly, in one aspect, the present invention provides a polypeptide operable in association with at least a second polypeptide to generate a polypeptide complex possessing enzyme activity in the presence of inhibitors of the enzyme activity.

In a preferred embodiment, the polypeptide comprises one or more amino acid sequence changes which reduce the susceptibility of the polypeptide complex to inhibitors of the enzyme activity.

In a related aspect, the present invention provides a reporter system comprising a first reporter component comprising a first polypeptide subunit and a second reporter component comprising a second polypeptide subunit, the first subunit and second subunit being capable of associating to generate an active polypeptide complex having enzyme activity which is capable of generating a detectable signal, said association being mediated by binding of the first and second reporter components to an analyte of interest;

wherein the first polypeptide subunit and/or the second polypeptide subunit comprise one or more amino acid sequence changes which reduce the susceptibility of the active polypeptide complex to inhibition of said enzyme activity by an inhibitor.

In specific embodiments, the invention provides for polypeptide constructs for use as reporter fragment pair domains based upon point mutations, break points, and deletions in the native amino acid sequence of TEM-1 Beta-lactamase that confer upon the resulting fragments the desired characteristics required for use in in vitro homogeneous FEC assays. These desired characteristics include, but are not limited to those that allow Beta-lactamase based FEC assays in homogeneous format in the presence of one or more blood serum components. In particular embodiments, these blood serum components include inhibitors of Beta-lactamase.

In preferred embodiments, the invention provides for specific mutations that may be advantageously introduced into fragment pair constructs to enhance the solubility, decrease aggregation, improve performance in the presence of Beta-lactamase inhibitors, and in other ways adapt the fragment pair to use in homogeneous in vitro FEC assays. The effects of these mutations may confer multiple advantages upon the reporter, linker, or interactor domains either alone (when used in conjunction with an operable pair member not of the invention) or in combination with operable pair members of the invention. These advantages include operability in vitro or in homogeneous assays conditions that may include inhibitors of Beta-lactamase or other factors present in sera or other biological samples that detract from the utility of FEC in such conditions.

In identifying the specific mutations (amino acid sequence changes) the notation followed herein (except where context demands otherwise) utilizes the one-letter code for amino acids in conjunction with positional information from a native Beta-lactamase amino acid sequence. For example, V74T indicates Valine at native position 74 (indicated in the accompanying sequence or at homologous positions in alternative sequence numbering schemes) is replaced by Threonine, and so forth. For consistency and convenience, the numbering of the native sequence will be retained even when referring to a sequence or reporter fragment of substantially fewer amino acids. Thus, F230Y refers to the substitution of F at position 230 with Y in reference to the full native sequence numbering even though the substitution may occur within a shorter, e.g. α or ω fragment of the present invention.

In particular embodiments, mutations of a native Beta-lactamase sequence of the invention are provided in conjunction with α and ω fragments formed by a breakpoint between the junction of Glycine at position 196 and Glutamic Acid at position 197 in the numbering of the accompanying sequence listings or at homologous positions in any alternative sequence numbering scheme. Other breakpoints giving rise to alternative α or ω fragments useful in conjunction with the mutations provided herein are within the routine skill of the relevant artisan.

The invention provides for α or ω fragments of TEM-1 Beta-lactamase comprising single or multiple amino acid sequence changes selected from the following: M69L; M69I; V74T; M182T; 1208T; M211Q; F230Y; and N276D. These mutations may be present individually or in combination. Specific combinations of mutations relative to the native sequence may be present wholly within a single reporter domain or reporter fragment pair member. Specific mutations or combinations of mutations may be present in a single reporter domain useful in FEC assays, or in two, complementary reporter domains or reporter fragment pair members, depending upon the assay desired and the operability of the mutations in meeting the requirements of an assay within the scope of the invention. Particular combinations of single amino acid mutations within the scope of the invention include: M69L with M182T in an α fragment reporter domain; M69I with M182T in an α fragment reporter domain; and N276D in a ω fragment reporter domain. These altered reporter domain sequences may, of course, be incorporated into appropriate reporter fragment members to constitute an operable member of an FEC assay. It is within the scope of the invention that the interactor domains may be any such domains that one of skill in the art may desire, linked to one or more of the altered reporter domains of the invention to constitute an operable member or operable pairs of members of an FEC assay. As one of skill in the art will appreciate, these constructs may be operable, and therefore be of use in vivo or in vitro. In vivo uses of the inhibitor resistant constructs of the present invention are specifically contemplated.

In further exemplary embodiments, a reporter fragment member of the invention comprises an isolated or purified polypeptide of a sequence selected from the group consisting of SEQ ID NOs: 2, 4, 6, 8 10, 12, 14, 16, 18, 20, 21, 22, 23, 24, 67, 69, 71, 73, 75, 77, 81 and 83. In some preferred embodiments, a reporter fragment of the invention consists essentially of an isolated or purified polypeptide of a sequence selected from the group consisting of SEQ ID NOs: 2, 4, 6, 8 10, 12, 14, 16, 18, 20, 21, 22, 23, 24, 67, 69, 71, 73, 75, 77, 81 and 83. In specific, preferred embodiments, a reporter fragment of the invention consists of an isolated or purified polypeptide of a sequence selected from the group consisting of group consisting of SEQ ID Nos: 2, 4, 6, 8 10, 12, 14, 16, 18, 20, 21, 22, 23, 24, 67, 69, 71, 73, 75, 77, 81 and 83.

The invention also provides isolated or purified specific fragment pairs comprising a pairing of reporter fragment pairs, wherein the pairing comprises at least one polypeptide selected from the group consisting of SEQ ID Nos: 2, 4, 6, 8 10, 12, 14, 16, 18, 20, 21, 22, 23, 24, 67, 69, 71, 73, 75, 77, 81 and 83. In specific embodiments, the polypeptides of the invention feature a substantial rearrangement of sequence, as exemplified by the amino acid sequence of SEQ ID NO: 21, which describes the construct labelled PB15. Further embodiments include reporter fragments derived from such rearranged basic sequences such as those exemplified in SEQ ID NOs: 23 and 24.

In keeping with the invention, linker domains may be added to the reporter fragments of the invention. Moreover, interactor domains may be joined to the reporter fragments of the invention with or without intervening linker domains. The number of linker domains, for example, G₄S domains (i.e. GGGGS sequence repeats) may be 0, 1, 2, 3, 4, 5, 6, or more and may be specified as (G₄S)n where n may be 0, 1, 2, 3, 4, 5, 6, or higher integer. The selection of an appropriate linker domain sequence and/or number of linker domain repeats for any particular interactor and reporter domain construct and fragment pairing is within the routine level of experimentation by the skilled artisan.

The reporter fragments of the invention include isolated and purified reporter fragments that may be operably constituted into a homogeneous in vitro FEC assay.

In additional aspects, the invention provides nucleic acids encoding the polypeptide embodiments of the invention, such as a nucleic acid comprising or consisting of a nucleotide sequence selected from the sequences shown in SEQ ID NOs: 5, 7, 9, 11, 13, 15, 17, 19, 66, 68, 70, 72, 74, 76, 80 and 82.

The invention further provides methods of making, purifying, isolating, and using the reporter fragments and in vitro FEC assays components described herein.

The invention further provides homogeneous in vitro FEC assays comprising isolated and purified polypeptides and reporter fragment pairs of the invention.

Accordingly, in another aspect, the present invention provides a method of assaying for the presence of an analyte, the method comprising the steps of:

(a) obtaining a sample to be tested for the presence of an analyte of interest;

(b) obtaining a purified first reporter fragment pair member comprising an interactor domain with affinity for the analyte;

(c) obtaining a purified second reporter fragment pair member comprising an interactor domain with affinity for the analyte of interest and operable in reconstituting a reporter enzyme activity upon association with the first reporter fragment pair member through the affinities of the interactor domains of the first and second reporter fragment pair members with the analyte of interest; and

(d) providing assay conditions in vitro sufficient to allow the first and second reporter fragment pair members to associate through the affinity of the interactor domains with the analyte, wherein reconstitution of the reporter enzyme activity indicates the presence of the analyte in the sample.

In a related aspect, the present invention also provides a method of determining the presence of an analyte of interest in a sample which method comprises contacting the sample with a reporter system of the invention and detecting the presence or absence of enzyme activity resulting from the association of the first and second polypeptide subunits.

In another related aspect, the present invention provides the use of a reporter system of the invention for determining the presence of an analyte of interest in a sample.

The invention also provides specific reporter fragment pairs adapted to perform homogeneous in vitro assays for particular analytes. In one embodiment, the analytes are divalent metal cations such as Ni²⁺, Zn²⁺, or Co²⁺. In yet another embodiment, the analyte is an antibody. The antibody analyte may be a monoclonal antibody or other, suitable antibody, depending upon the assay of interest. In further embodiments, the analyte is an antigen. In additional embodiments, the analyte is one or more polynucleotides or a specific sequence. Such polynucleotides may or may not be methylated, and their methylation status may be information gained from use of particular embodiments of the invention.

In additional embodiments, the invention includes kits for performing the assays of the invention, or kits including comprising materials, reagents, and instructions for making the assay components of the invention or subsequent use of the assays of the invention.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF DRAWINGS

The following drawings form part of the present specification and are included to further demonstrate certain aspects of the present invention. The invention may be better understood by reference to one or more of these drawings in combination with the detailed description and examples of the invention presented herein.

FIG. 1. Schematic drawing of FEC assay and its basic components.

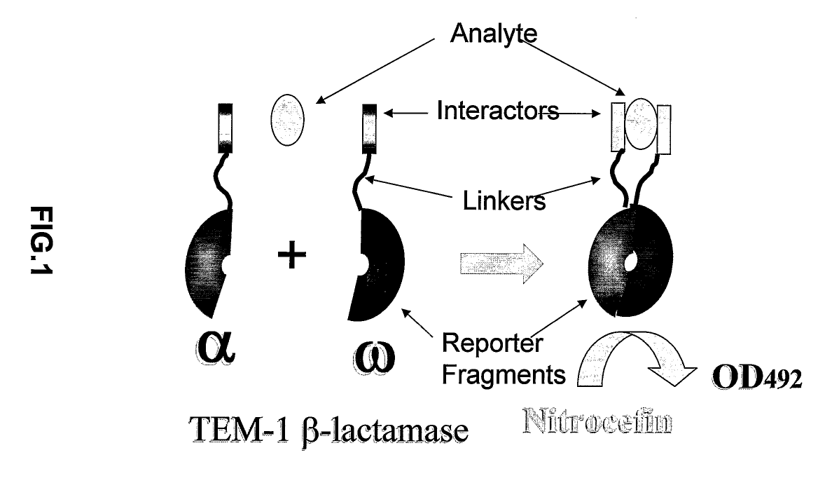

FIG. 2. Schematic of homogeneous in vitro FEC assay for divalent metal cations.

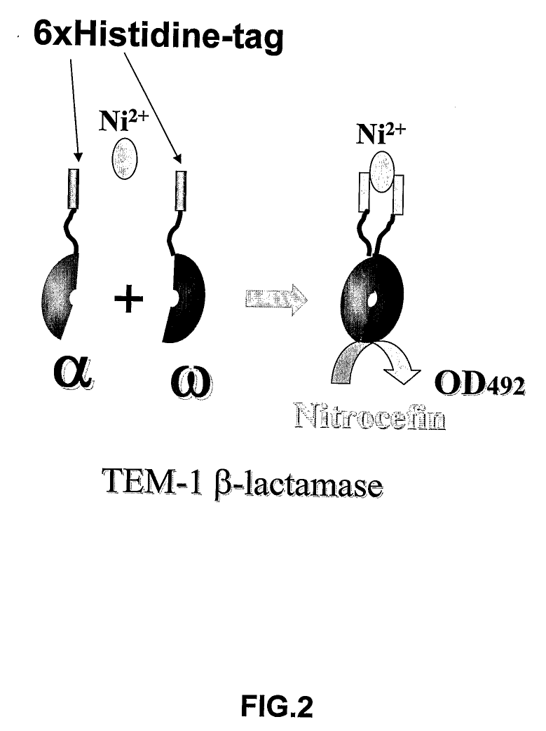

FIG. 3. Performance of homogeneous in vitro FEC assays for divalent cations employing wild type or mutant Beta-lactamase reporter constructs.

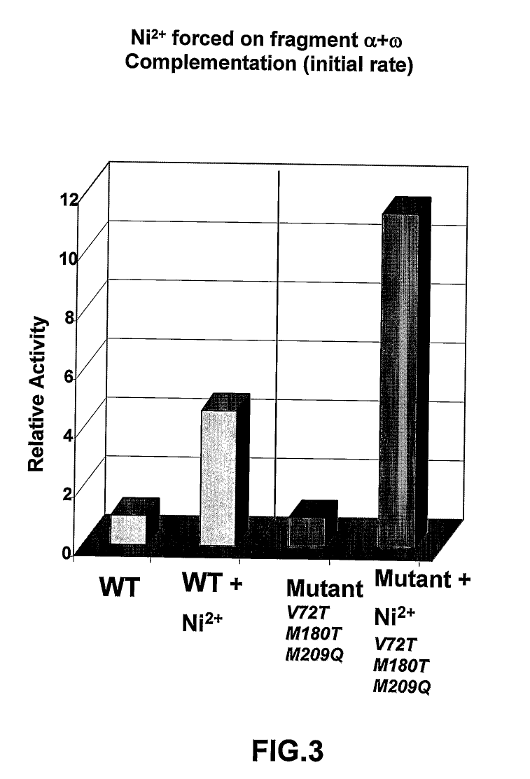

FIG. 4. 10 minute OD₄₉₂ tests on homogeneous in vitro FEC assays for Ni²⁺.

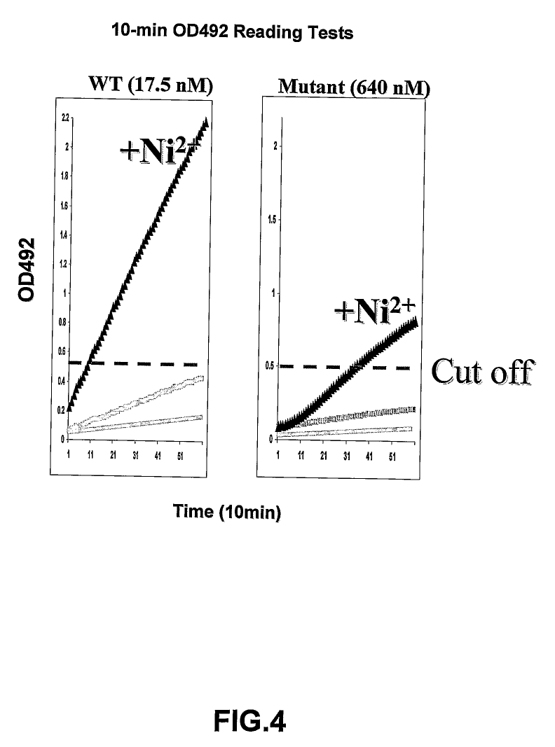

FIG. 5. Schematic of homogeneous in vitro FEC assay for an anti-histidine tag monoclonal antibody.

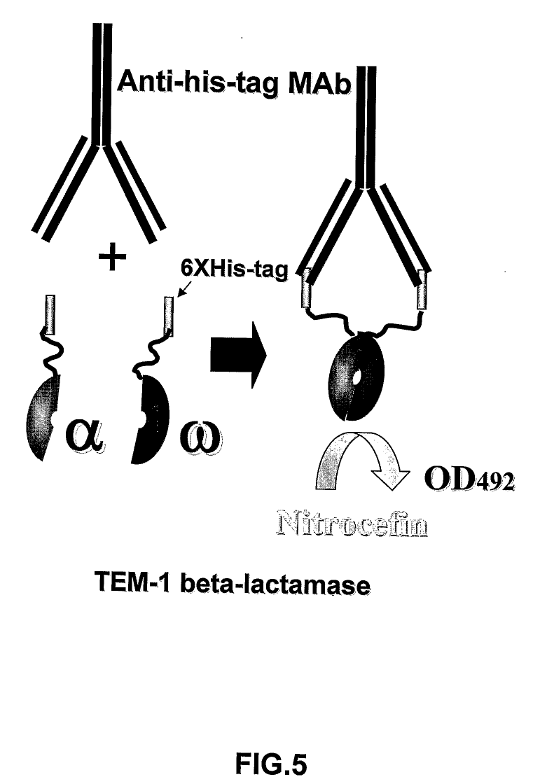

FIG. 6. Performance (absorbance at OD₄₉₂) of homogeneous in vitro FEC assays for an anti-histidine tag monoclonal antibody.

FIG. 7. Purification of protein fragments of the invention. The panel on the left of the figure is gel electrophoresis of pre- and post-induction expression of the α Beta-Lactamase enzyme fragment and of the affinity purified material. The chromatogram labelled “Affinity purified” and the chromatogram labelled “Gel filtration profile of α fragment both illustrate embodiments of purified enzyme fragment. Similar results are obtained for other fragments of the invention.

FIG. 8. Absorbance reading at 492 nm of the progress of an FEC in vitro assay using Beta-Lactamase enzyme fragments responsive to Ni²⁺ as analyte and equimolar amounts of α and ω Beta-Lactamase fragments of the invention.

FIG. 9. Demonstration of the intensity of color change over an in vitro assay for Ni²⁺ in multiple well plate format. Time points are non-linear. Intensity of color in each well is demonstrated in the presence and absence of analyte (wells A1-A8 and B1-B8, respectively) and substrate only (wells C₁-C₈).

FIG. 10. Characteristics of assays of mutant constructs. Units are mOD min⁻¹.

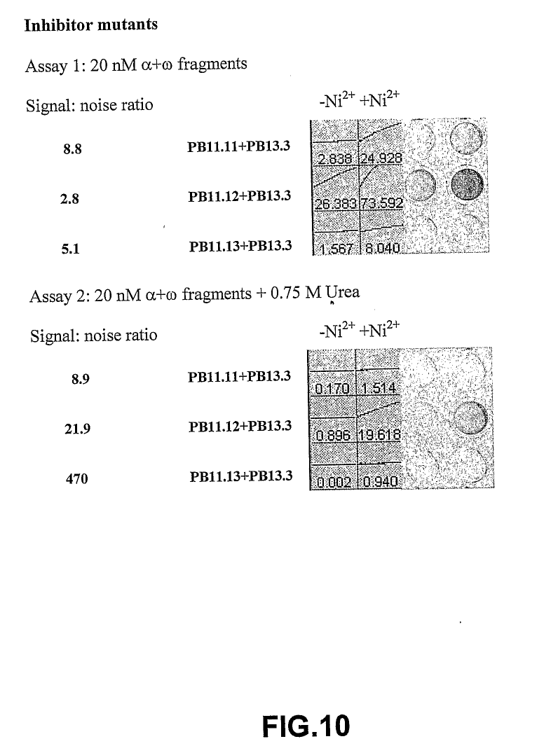

FIG. 11. Characteristics of assays of constructs for HSV. Units are mOD min⁻¹.

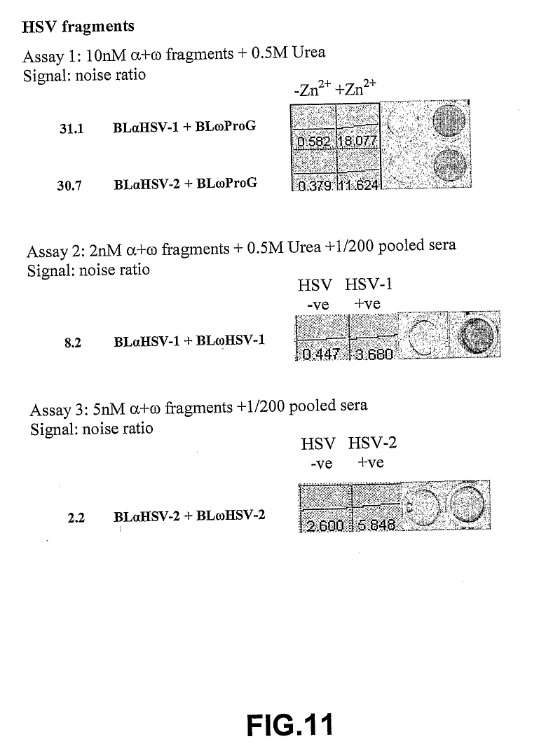

FIG. 12. Graph showing relative resistance of optimal inhibitor resistant mutant TEM-33M182TN276D (PB11.12+PB13.3) as compared to wild-type TEM-1 (PB11+PB13) FEC.

FIG. 13. Graph showing results of beta-lactamase inhibitor spiked serum assays of optimal inhibitor resistant mutant TEM-33M182TN276D (PB11.12+PB13.3) as compared to wild-type TEM-1 (PB11+PB13) FEC and full-length TEM-1 Beta-lactamase.

FIG. 14. Graph showing characteristics of HSV-1 assay using BLαProG/BLω-HSV1. Cut off=1.20. Sensitivity=98% for HSV1+ve. Specificity=100% for HSV2+ve. Specificity=100% for HSV-ve.

FIG. 15. Graph showing characteristics of HSV-2 assay using BLαProG/BLω-HSV2. Cut off=1.16. Sensitivity=94% for HSV2+ve. Specificity=100% for HSV1+ve. Specificity=100% for HSV-ve.

FIG. 16. Graph showing characteristics of HSV-1 assay using BLαHSV1/BLω-ProG. Cut off=1.15. Sensitivity=98% for HSV1+ve. Specificity=100% for HSV2+ve. Specificity=96% for HSV-ve.

FIG. 17. Graph showing characteristics of HSV-2 assay using BLαHSV1/BLω-ProG. Cut off=1.20. Sensitivity=98% for HSV2+ve. Specificity=100% for HSV1+ve. Specificity=96% for HSV-ve.

FIG. 18. Graph showing typical rates of nitrocefin hydrolysis (monitored at 492 nm, following a 15 minute pre-incubation period) in the presence of either normal (control) or HSV-2 high-positive patient serum. Average rates of hydrolysis are 0.85 mOD min⁻¹ (HSV negative) and 5.14 mOD min⁻¹.

FIG. 19. Cartoon representation of β-lactamase-based FEC and the sequences of HSV-1 and HSV-2 specific antigenic peptides. (a) Enzyme fragments, α and ω, are linked to analyte binding moieties such as a disease-specific antigenic peptide (P) and one domain of protein G. In the presence of analyte (disease-specific antibody), the fragments are forced into close proximity (right), thereby initiating the hydrolysis of nitrocefin, visible as a color change from yellow to red. (b) Truncated glycoprotein amino acid sequence of HSV-1 and HSV-2 antigenic peptides. The underlined and bold types represent the immunodominant regions.

FIG. 20. Schematic representation of FEC fragments used in this study. BL refers to β-lactamse alpha ( [symbol] blue) and omega ( [symbol] green) fragments. Analyte binding moieties (red), ProG, HSV-P1, and HSV-P2 refer to the protein G domain, and HSV type-specific antigenic peptides for HSV-1 and HSV-2, respectively. Histidine tags are shown in grey. (Gly₄Ser)₃ linkers were used to join the enzyme fragments to the analyte binding moieties.

### BRIEF DESCRIPTION OF SEQUENCE LISTINGS

SEQ ID NO: 1. PB11, nucleotide sequence encoding the alpha fragment construct of native Beta-lactamase sequence.

SEQ ID NO: 2. PB11, amino acid sequence of alpha fragment construct of native Beta-lactamase.

SEQ ID NO: 3. PB13, nucleotide sequence encoding the omega fragment construct of native Beta-lactamase sequence.

SEQ ID NO: 4. PB13, amino acid sequence of omega fragment construct of native Beta-lactamase.

SEQ ID NO: 5. PB11.2, nucleotide sequence encoding the alpha fragment construct of Beta-lactamase sequence containing substitutions of V74T and M182T.

SEQ ID NO: 6. PB11.2, amino acid sequence of the alpha fragment construct of Beta-lactamase sequence containing substitutions of V74T and M182T.

SEQ ID NO: 7. PB11.11 alpha fragment construct of Beta-lactamase sequence containing substitution M69L.

SEQ ID NO: 8. PB11.11 amino acid sequence of the alpha fragment construct of Beta-lactamase sequence containing substitution M69L.

SEQ ID NO: 9. PB11.12 alpha fragment construct of Beta-lactamase sequence containing substitutions M69L and M182T.

SEQ ID NO: 10. PB11.12 amino acid sequence of the alpha fragment construct of Beta-lactamase sequence containing substitution M69L and M182T.

SEQ ID NO: 11. PB13.1 nucleotide sequence encoding the omega fragment construct of Beta-lactamase sequence containing substitution of M211Q.

SEQ ID NO: 12. PB13.1 amino acid sequence of the omega fragment construct of Beta-lactamase sequence containing substitution of M211Q.

SEQ ID NO: 13. PB11.13 nucleotide sequence encoding the alpha fragment construct of Beta-lactamase sequence containing substitutions M691 and M182T.

SEQ ID NO: 14. PB11.13 amino acid sequence of the alpha fragment construct of Beta-lactamase sequence containing substitution M69I and M182T.

SEQ ID NO: 15. PB11.4, nucleotide sequence encoding alpha fragment of native Beta-lactamase including (G4S)₃ linker domain.

SEQ ID NO: 16. PB11.4, amino acid sequence of alpha fragment of native Beta-lactamase including (G4S)₃ linker domain.

SEQ ID NO: 17. PB13.2, nucleotide sequence encoding omega fragment of native Beta-lactamase including (G4S)₃ linker domain.

SEQ ID NO: 18. PB13.2, amino acid sequence of a omega fragment of native Beta-lactamase including (G4S)₃ linker domain.

SEQ ID NO: 19. PB13.3 omega fragment construct of Beta-lactamase containing substitution N276D.

SEQ ID NO: 20. PB13.3 amino acid sequence of omega fragment of native Beta-lactamase containing substitution N276D.

SEQ ID NO: 21. PB15, amino acid sequence of rearranged construct of Beta-lactamase.

SEQ ID NO: 22. PB15.3, amino acid sequence of rearranged construct of Beta-lactamase and including substitutions of M182T, 1208T, and F230Y.

SEQ ID NO: 23. PB7.2, amino acid sequence of alpha fragment of PB15.3.

SEQ ID NO: 24. PB9.1, amino acid sequence of omega fragment of PB15.3.

SEQ ID NO: 25 through SEQ ID NO: 65 are synthetic primers as described in Tables 1 and 5.

SEQ ID NO: 66 P1-1 trunc, HSV-1 truncated antigen nucleotide sequence.

SEQ ID NO: 67 P1-1 trunc, HSV-1 truncated antigen amino acid sequence.

SEQ ID NO: 68 BLα HSV-1 alpha fragment construct nucleotide sequence.

SEQ ID NO: 69 BLα HSV-1 alpha fragment construct amino acid sequence.

SEQ ID NO: 70 BLω, HSV-1 omega fragment construct nucleotidesequence.

SEQ ID NO: 71 BLω, HSV-1 omega fragment construct amino acid sequence.

SEQ ID NO: 72 P2-1 trunc, HSV-2 truncated antigen nucleotide sequence.

SEQ ID NO: 73 P2-1 trunc, HSV-2 truncated antigen amino acid sequence.

SEQ ID NO: 74 BLα HSV-2 alpha fragment construct nucleotide sequence.

SEQ ID NO: 75 BLα HSV-2 alpha fragment construct amino acid sequence.

SEQ ID NO: 76 BLω HSV-2 omega fragment construct nucleotide sequence.

SEQ ID NO: 77 BLω HSV-2 omega fragment construct amino acid sequence.

SEQ ID NO: 78 Protein G nucleotide sequence.

SEQ ID NO: 79 Protein G amino acid sequence.

SEQ ID NO: 80 BLαProG, protein G alpha fragment construct nucleotide sequence.

SEQ ID NO: 81 BLαProG, protein G alpha fragment construct amino acid sequence.

SEQ ID NO: 82 BLωProG, protein G omega fragment construct nucleotide sequence.

SEQ ID NO: 83 BLωProG, protein G omega fragment construct amino acid sequence.

SEQ ID NO: 84 Amino acids 92 to 148 of gG1 of HSV1.

SEQ ID NO: 85 Amino acids or 551 to 641 of gG2 of HVS2.

### DETAILED DESCRIPTION

Unless defined otherwise, all technical and scientific terms used herein have the same meaning as commonly understood by one of ordinary skill in the art (e.g. in cell biology, chemistry and molecular biology). Standard techniques used for molecular and biochemical methods can be found in Sambrook et al., Molecular Cloning: A Laboratory Manual, 3^{rd} ed. (2001) Cold Spring Harbor Laboratory Press, Cold Spring Harbor, N.Y. and Ausubel et al., Short Protocols in Molecular Biology (1999) 4^{th} Ed, John Wiley & Sons, Inc.—and the full version entitled Current Protocols in Molecular Biology).

The following detailed descriptions of particular embodiments and examples are offered by way of illustration and not by way of limitation. Unless contraindicated or noted otherwise, in these descriptions and throughout this specification, the terms “a” and “an” mean one or more. Similarly the term “or” means “and/or”.

By “comprising” is meant including, but not limited to, whatever follows the word “comprising”. Thus, use of the term “comprising” indicates that the listed elements are required or mandatory, but that other elements are optional and may or may not be present. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range.

### Reporter Components and Reporter Polypeptide Fragments

The present invention provides reporter components that comprise or consist of polypeptides that are adapted for use as isolated and purified components of homogeneous in vitro FEC assays. The reporter components form part of a reporter system. As discussed above, the reporter system in FEC assays includes two or more polypeptide fragments that when they associate, form a reporter protein complex that can give rise to a detectable signal. Accordingly, each reporter component includes at least one such polypeptide fragment or subunit (herein termed a “reporter fragment”). For example, in the case of a system based on beta lactamase, a first reporter component may comprise an alpha fragment of the beta lactamase and a second reporter component may comprise an omega fragment of the beta lactamase such that when the two reporter components associate under assay conditions, the resulting complex has beta lactamase activity. A combination of a first reporter component and a second reporter component which together can give rise to detectable enzyme activity when their respective reporter polypeptide fragments (also termed “subunits”) are brought into association, is termed herein a “reporter component pair”, the respective reporter polypeptide fragments being collectively termed a “reporter fragment pair”.

Examples of reporter polypeptides that can be used as the basis for reporter fragments and systems include beta-lactamase (e.g. TEM-1 beta-lactamase: EC: 3.5.2.6) beta-galactosidase and bioluminescent proteins such as luciferases (e.g. firefly and Renilla luciferases) and fluorescent proteins including green fluorescent proteins. The reporter polypeptides are typically split into two fragments which when they associate can reconstitute the activity of the original full length polypeptide. For example, beta-lactamase and beta-galactosidase are typically split into two fragments, an alpha and an omega fragment. Suitable breakpoints in the amino acid sequences of these various proteins to generate the two fragments have been described previously.

Reporter polypeptides/fragments are selected so that they are suitable for in vitro use (for the avoidance of doubt, in the present context the term in vitro means that the assays take place outside of living cells). The reporter polypeptides are typically variants of wild type sequences that have amino acid changes that improve their suitability for in vitro use, for example to enhance stability, improve solubility and/or reduce aggregation. In a particularly preferred embodiment, the reporter polypeptides/fragments have reduced sensitivity, compared to wild type polypeptides, to inhibitors, such as enzyme inhibitors, of the activity of the polypeptide required for reporter function, e.g. beta lactamase activity. Such inhibitors include compounds found in samples, e.g. biological samples such as blood and serum samples, that it is desired to test for the presence of analytes of interest. Particular examples of such inhibitors are inhibitors of beta-lactamase activity found in blood as a result of administering antibiotics to individuals from whom the blood samples are taken.

Under assay conditions, the association of the reporter fragments to form an active complex having reporter activity is typically mediated by the interaction between other regions of the reporter components and a target analyte. Thus, the reporter components typically comprise an interactor moiety or domain.

Interactor domains have binding specificity for a target analyte of interest. Interactor domains include, for example, peptides, glycoproteins, polysaccharides, antigens, antibodies and antigen-binding fragments of antibodies such as complementarity determining regions (CDRs). Antigens include antigens derived from pathogens, such as viral or bacterial antigens. Antibodies/CDRs include sequences that bind to antigens derived from pathogens, such as viruses or bacteria. Particular examples of interactor domains include the IgG-binding domain of Protein G, and Herpes Simple Virus antigens (particularly preferred versions of which are truncated glycoprotein G1 envelope proteins from HSV1 or truncated glycoprotein G2 envelope proteins from HSV2, such as amino acids 92 to 148 of gG1 or 551 to 641 of gG2).

Interactor domains may be joined directly to the reporter fragments or via a linker. Suitable linker domains include peptides, such as glycine rich repeat sequences (e.g. G₄S repeat sequences—i.e. GGGGS sequence repeats). The number of linker domains, for example, G₄S domains may be 1, 2, 3, 4, 5, 6, or more. In one embodiment, the number of glycine rich repeat sequences is preferably 2 or 3, particularly where the interactor domain is a polypeptide having fewer than 150 or 100 amino acids. Where larger interactor domains are used, it may be desirable to increase linker length.

In one embodiment, the linker domain is flexible. In another embodiment the linker domain is rigid.

The selection of an appropriate linker domain sequence and/or number of linker domain repeats for any particular interactor and reporter fragment construct and fragment pairing is within the routine level of experimentation by the skilled artisan.

Reporter fragment polypeptides, linker domains and interactor domains may be joined by covalent or non-covalent means to form a reporter component.

In one embodiment, the linker domains and interactor domains are polypeptides. Thus the reporter component may be a single polypeptide.

In another embodiment, the reporter fragments are linked to interactor domains by conjugation e.g. covalent coupling via, for example, thiol-thiol, amine-carboxyl or amine-aldehyde functional groups. Particular examples include cross-linking of polypeptides to glycoproteins via the carbohydrate groups; cross-linking via primary amines (found at the N-terminus and on lysine residues) e.g. using heterobifunctional cross linkers with an amine reactive group and a sulfhydryl reactive group; cross-linking via carboxyl groups (found at C-terminus and as side groups on glutamic acid and aspartic acid residues); cross-linking via free sulfhydryl groups; and disulphide exchange.

Non-covalent methods include avidin-biotin systems and hybridization of oligonucleotide-protein conjugates.

The present invention in one aspect provides isolated or purified peptide and protein reporter fragments and reporter components, such as those that consist of, or consist essentially of, or comprise the amino acid sequences of the enzyme peptides disclosed herein. Exemplary sequences of the invention are provided in the figures or sequence listings. The peptide sequences provided will be referred herein as the reporter fragments or reporter pair members/components of the assays described in the present invention.

As used herein, a peptide/polypeptide/protein is said to be “isolated” or “purified” when it is substantially free of cellular material or free of chemical precursors or other chemicals. The peptides of the present invention may be purified to homogeneity or other degrees of purity. The level of purification will be based on the intended use. The intended use in the present invention is as operable components of homogenous in vitro FEC assays for specific analytes.

In some uses, “substantially free of cellular material” includes preparations of the peptide having less than about 30% (by dry weight) of other proteins (i.e., contaminating protein), less than about 20% other proteins, less than about 10% other proteins, or less than about 5% other proteins. When the peptide is recombinantly produced, it may also be substantially free of culture medium, i.e., culture medium represents less than about 20% of the volume of the protein preparation.

The language “substantially free of chemical precursors or other chemicals” includes preparations of the peptide in which it is separated from chemical precursors or other chemicals that are involved in its synthesis. In one embodiment, the language “substantially free of chemical precursors or other chemicals” includes preparations of the enzyme peptide having less than about 30% (by dry weight) chemical precursors or other chemicals, less than about 20% chemical precursors or other chemicals, less than about 10% chemical precursors or other chemicals, or less than about 5% chemical precursors or other chemicals.

The isolated reporter fragment and polypeptide reporter components may be purified from cells that have been altered to express it (recombinant), or synthesized using known protein synthesis methods. For example, a nucleic acid molecule encoding the enzyme peptide is cloned into an expression vector, the expression vector introduced into a host cell and the protein expressed in the host cell. Suitable host cells are described in more detail below. The protein may then be isolated from the cells by an appropriate purification scheme using appropriate protein purification techniques. Exemplary techniques of the invention are described in detail in the Examples set out below.

In one aspect, the present invention provides proteins consisting of the amino acid sequences provided. A protein consists of an amino acid sequence when the amino acid sequence is the final amino acid sequence of the protein. In an additional aspect, the present invention further provides proteins that consist essentially of the amino acid sequences provided. A protein consists essentially of an amino acid sequence when such an amino acid sequence is present with only a few additional amino acid residues that do not alter the functional characteristics of the proteins of the invention. In yet a further aspect, the present invention provides proteins that comprise the amino acid sequences provided. A protein comprises an amino acid sequence when the amino acid sequence is at least part of the final amino acid sequence of the protein. In such a fashion, the protein may be only the peptide or have additional amino acid molecules, such as amino acid residues (contiguous encoded sequence) that are associated with it or heterologous amino acid residues or peptide sequences. Such a protein may have a few additional amino acid residues or may comprise several hundred or more additional amino acids. A brief description of how various types of these proteins may be made or isolated is provided below.

The peptides of the present invention may be attached to heterologous sequences to form chimeric or fusion proteins. Such chimeric and fusion proteins may comprise an enzyme peptide operatively linked to a heterologous protein having an amino acid sequence not substantially homologous to the enzyme peptide. “Operatively linked” indicates that the enzyme peptide and the heterologous protein are fused such that the operability of each is not destroyed. The heterologous protein may be fused to the N-terminus or C-terminus of the enzyme peptide.

A chimeric or fusion protein may be produced by standard recombinant DNA techniques. For example, DNA fragments coding for the different protein sequences are ligated together in-frame in accordance with conventional techniques. In another embodiment, the fusion gene may be synthesized by conventional techniques including automated DNA synthesizers. Alternatively, PCR amplification or ligation of gene fragments may be carried out using anchor primers which give rise to complementary overhangs between two consecutive gene fragments which may subsequently be annealed and re-amplified to generate a chimeric gene sequence (see Ausubel et al., Current Protocols in Molecular Biology, 1998). Moreover, many expression vectors are commercially available that already encode a fusion moiety. An enzyme peptide-encoding nucleic acid may be cloned into such an expression vector such that the fusion moiety is linked in-frame to the enzyme peptide, which is one means by which the fusion protein is made without destroying the operability of each component.

### Identification of Homologous Positions in Alternative Sequence Numbering Schemes

To determine homologous positions in comparing two amino acid or nucleotide sequences, the sequences are aligned for optimal comparison purposes (e.g., gaps may be introduced in one or both of a first and a second amino acid or nucleotide sequence for optimal alignment and non-homologous sequences may be disregarded for comparison purposes). In a preferred implementation, at least 30%, 40%, 50%, 60%, 70%, 80%, or 90% or more of the length of a reference sequence is aligned for comparison purposes. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position and the position is homologous in the two. The percent identity between the two sequences is a function of the number of identical positions shared by the sequences, taking into account the number of gaps, and the length of each gap, which need to be introduced for optimal alignment of the two sequences.

The comparison of sequences and determination of percent identity and similarity between two sequences may be accomplished using a mathematical algorithm. (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991).

For example, pairwise alignments and levels of sequence identity and homology may be determined using the BestFit program in the GCG software package. The percent identity between two amino acid sequences may be determined using the algorithm of Needleman and Wunsch (J. Mol. Biol. 48:444-453 (1970)), which has been incorporated into the GAP program in the GCG software package. The algorithm is typically employed using either a Blossom 62 matrix or a PAM250 matrix, and a gap weight of 16, 14, 12, 10, 8, 6, or 4 and a length weight of 1, 2, 3, 4, 5, or 6. The percent identity between two nucleotide sequences may be determined using the GAP program (Devereux, J., et al., Nucleic Acids Res. 12(1):387 (1984)) with a NSWgapdna.CMP matrix and a gap weight of 40, 50, 60, 70, or 80 and a length weight of 1, 2, 3, 4, 5, or 6.

Thus, a substitution at a position in an amino acid sequence or peptide is indicated by the one letter designation for the amino acid, followed by the position number of the relevant non-substituted sequence or peptide, followed by one or more one letter designations for replacement amino acids. For example, substitution of Threonine for Valine at position 72 of the α fragment of TEM-1 Beta-lactamase as indicated in the accompanying sequence listings and figures would be designated as V74T. Similar designations will be clear from the context and further details provided herein.

### Amino Acid Modifications to Improve Reporter Functionality In Vitro

Reporter polypeptide fragments for use in the assays of the invention generally comprise amino acid sequence changes or modifications that improve the suitability of the reporter polypeptide for use in such assays. In particular, in a preferred embodiment, at least one of the reporter polypeptide fragments comprises a variation in or modification to its amino acid sequence which renders a reconstituted active reporter polypeptide complex less susceptible to inhibition by substances that are inhibitors of the unmodified (e.g. wild type) amino acid sequence.

Such variants typically contain one or more non-conservative amino acid substitutions, deletions, insertions, inversions, or truncation or a substitution, insertion, inversion, or deletion in a critical residue or critical region. Variants of altered function may also contain substitution of similar amino acids that result in no change or an insignificant change in function. In one embodiment, variants contain 1, 2, 3, 4 or 5 amino acid changes.

The present invention provides modified Beta-lactamase peptide sequences comprising single amino acid substitutions or multiple substitutions in combination that are especially adapted for use in homogeneous in vitro FEC assays as further exemplified herein. Specific examples of such substitutions are substitutions at amino acid position 69 (preferably M69L or M69I) in the alpha fragment, which reduces inhibition by beta-lactamase inhibitors and substitutions at amino acid position 276 (preferably N276D) in the omega fragment, which also reduces inhibition by beta-lactamase inhibitors. Other examples are selected from substitutions at one or more of amino acid positions 74, 182, 208, 211 and 230 (preferably one or more of V74T, M182T, 1208T, M211Q and F230Y).

Further examples are mutations of the following hydrophobic amino acid residues to any residue which is considered to be less hydrophobic may give rise to an improved assay: V44, Y46, L49, L51, F66, V74 L81, F151, P183, V184, A187, L190, L194, L198, L199, L207, I208, W210, M211, A232, I247, A249, P257, I260, I261, I262, I263, Y264, I282, L286 (particularly residues that are in bold type and underlined).

The amino acid numbering given is with reference to the wild-type TEM-1 beta lactamase sequence shown in SEQ ID NOS: 1 and 3. However, it will be appreciated that the modifications can also be applied to homologous beta lactamase sequences at the equivalent positions (see preceding section for determination of equivalent positions by sequence alignments).

Modified reporter fragments having improved properties for use in in vitro assays, such as homogeneous assays, can be obtained using various techniques. For example, sequence changes may be introduced by site-directed mutagenesis. The selection of suitable sites may, for example, be guided by the primary amino acid sequence and/or secondary/tertiary structural information included structural information determined by techniques such as x-ray crystallography or NMR. For example, the 3D structure of the active site of an enzyme can be used to assist in designing variants which have reduced susceptibility to inhibition (see for example, the crystal structure of TEM1 as described in Jelsch, C., F. Lenfant, et al., 1992, FEBS Lett 299(2): 135-42)

Modified reporter fragments having improved properties can also be obtained by techniques such as random mutagenesis or directed molecular evolution followed by selection of variants having the desired properties (e.g. by testing variants for enzyme activity in the presence of an inhibitor of the unmodified protein).

Additional modification useful in the present invention may include sequences containing amino acids other than the 20 amino acids commonly referred to as the 20 naturally occurring amino acids. Further modifications include, but are not limited to, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination.

Accordingly, peptides and constructs of the present invention also encompass derivatives or analogs in which a substituted amino acid residue is not one encoded by the genetic code, in which a substituent group is included, in which the mature enzyme peptide is fused with another compound, such as a compound to increase the half-life of the enzyme peptide (for example, polyethylene glycol), or in which the additional amino acids are fused to the mature enzyme peptide, such as a leader or secretory sequence or a sequence for purification of the mature enzyme peptide or a pro-protein sequence.

### Nucleic Acid Molecules

The present invention provides isolated nucleic acid molecules that encode an enzyme peptide, or protein of the present invention, including reporter polypeptide fragments and reporter components as described herein. In particular embodiments, the invention provides isolated nucleic acid molecules that encode an enzyme peptide or protein of the present invention as described in the figures and appended sequence listing, and various modifications or fragments thereof. Such nucleic acid molecules may consist of, consist essentially of, or comprise a nucleotide sequence that encodes one of the peptides or constructs of the present invention.

As used herein, an “isolated” nucleic acid molecule is one that is separated from other nucleic acid present in the natural source of the nucleic acid. Moreover, an “isolated” nucleic acid molecule, such as a transcript or cDNA molecule, is substantially free of other cellular material, or culture medium when produced by recombinant techniques, or chemical precursors or other chemicals when chemically synthesized. However, the nucleic acid molecule may be fused to other coding or regulatory sequences and still be considered isolated.

For example, recombinant DNA molecules contained in a vector are considered isolated. Further examples of isolated DNA molecules include recombinant DNA molecules maintained in heterologous host cells or purified (partially or substantially) DNA molecules in solution. Isolated RNA molecules include in vivo or in vitro RNA transcripts of the isolated DNA molecules of the present invention as well as novel fragments thereof. Isolated nucleic acid molecules according to the present invention further include such molecules produced synthetically.

Accordingly, the present invention provides nucleic acid molecules that consist of the nucleotide sequences provided. A nucleic acid molecule consists of a nucleotide sequence when the nucleotide sequence is the complete nucleotide sequence of the nucleic acid molecule. The present invention further provides nucleic acid molecules that consist essentially of the nucleotide sequences provided. A nucleic acid molecule consists essentially of a nucleotide sequence when such a nucleotide sequence is present with only a few additional nucleic acid residues in the final nucleic acid molecule.

The present invention further provides nucleic acid molecules that comprise the nucleotide sequences provided. A nucleic acid molecule comprises a nucleotide sequence when the nucleotide sequence is at least part of the final nucleotide sequence of the nucleic acid molecule. In such a fashion, the nucleic acid molecule may be only the nucleotide sequence or have additional nucleic acid residues, such as nucleic acid residues that are naturally associated with it or heterologous nucleotide sequences. Such a nucleic acid molecule may have a few additional nucleotides or may comprise several hundred or more additional nucleotides. A brief description of how various types of these nucleic acid molecules may be readily made or isolated is provided below.

The isolated nucleic acid molecules include, but are not limited to, the sequence encoding the enzyme peptide alone, the sequence encoding the mature peptide and additional coding sequences, such as a leader or secretory sequence (e.g., a pre-pro or pro-protein sequence), the sequence encoding the mature peptide, with or without the additional coding sequences, plus additional non-coding sequences, for example introns and non-coding 5′ and 3′ sequences such as transcribed but non-translated sequences that play a role in transcription, mRNA processing (including splicing and polyadenylation signals), ribosome binding and stability of mRNA. In addition, the nucleic acid molecule may be fused to a marker sequence encoding, for example, a peptide that facilitates purification.

Isolated nucleic acid molecules may be in the form of RNA, such as mRNA, or in the form DNA, including cDNA and genomic DNA obtained by cloning or produced by chemical synthetic techniques or by a combination thereof. The nucleic acid, especially DNA, may be double-stranded or single-stranded. Single-stranded nucleic acid may be the coding strand (sense strand) or the non-coding strand (anti-sense strand).

Allowing for the degeneracy of the genetic code, sequences are considered essentially the same as those set forth if they have at least about 50%, usually at least about 60%, more usually about 70%, most usually about 80%, preferably at least about 90% and most preferably about 95% of nucleotides that are identical to the nucleotide sequence of the invention. Sequences that are essentially the same as those set forth may also be functionally defined as sequences that are capable of hybridizing to a nucleic acid segment containing the complement of a polynucleotide under standard conditions. The term closely related sequence is used herein to designate a sequence with a minimum or 50% similarity with a polynucleotide or polypeptide with which it is being compared.

Naturally, the present invention also encompasses oligonucleotides that are complementary, or essentially complementary to the sequences of a polynucleotide of the invention. Nucleic acid sequences that are “complementary” are those that are capable of base-pairing according to the standard Watson-Crick complementary rules. As used herein, the term “complementary sequences” means nucleotide sequences that are substantially complementary, as may be assessed by the same nucleotide comparison set forth above, or as defined as being capable of hybridizing to the nucleic acid segment of a polynucleotide under relatively stringent conditions such as those described herein. Variants of the nucleic acids of the invention may be identified using methods well known in the art. These variants comprise a nucleotide sequence encoding a peptide that is typically 60-70%, 70-80%, 80-90%, and more typically at least about 90-95% or more homologous to the nucleotide sequence shown in the Figure sheets or a fragment of this sequence. Such nucleic acid molecules may readily be identified as being able to hybridize under moderate to stringent conditions, to the nucleotide sequence shown in the Figure sheets or a fragment of the sequence.

As used herein, and as well known to those of skill in the art, “high stringency conditions”, “moderate stringency conditions” and “low stringency conditions” for nucleic acid hybridizations are explained on pages 2.10.1-2.10.16 and pages 6.3.1-6 in Current Protocols in Molecular Biology (Ausubel, F. M. et al., “Current Protocols in Molecular Biology”, John Wiley & Sons, (1998)). The exact conditions which determine the stringency of hybridization depend not only on ionic strength (e.g., 0.2×SSC, 0.1×SSC), temperature (e.g., room temperature, 42 degrees Celsius, 68 degrees Celsius) and the concentration of destabilizing agents such as formamide or denaturing agents such as SDS, but also on factors such as the length of the nucleotide sequence, base composition, percent mismatch between hybridizing sequences and the frequency of occurrence of subsets of that sequence within other non-identical sequences. Thus, high, moderate or low stringency conditions can be determined empirically. By varying hybridization conditions from a level of stringency at which no hybridization occurs to a level at which hybridization is first observed, conditions which will allow a given sequence to hybridize (e.g., selectively) with the most similar sequences in the sample can be determined.

Exemplary conditions are also described in Krause, M. H. and S. A. Aaronson, Methods in Enzymology, 200:546-556 (1991). Also see Ausubel et al., “Current Protocols in Molecular Biology”, John Wiley & Sons, (1998), which describes the determination of washing conditions for moderate or low stringency conditions. Washing is the step in which conditions are usually set so as to determine a minimum level of complementarity of the hybrids. Generally, starting from the lowest temperature at which only homologous hybridization occurs, each degree Celsius by which the final wash temperature is reduced (holding SSC concentration constant) allows an increase by 1% in the maximum extent of mismatching among the sequences that hybridize. Generally, doubling the concentration of SSC results in an increase in Tm of about 17 degrees Celsius. Using these guidelines, the washing temperature can be determined empirically for high, moderate or low stringency, depending on the level of mismatch sought.

For example, a low stringency wash can comprise washing in a solution containing 0.2×SSC/0.1% SDS for 10 min at room temperature; a moderate stringency wash can comprise washing in a prewarmed solution (42 degrees Celsius) solution containing 0.2×SSC/0.1% SDS for 15 min at 42 degrees Celsius; and a high stringency wash can comprise washing in prewarmed (68 degrees Celsius) solution containing 0.1×SSC/0.1% SDS for 15 min at 68 degrees Celsius. Furthermore, washes can be performed repeatedly or sequentially to obtain a desired result as known in the art. Equivalent conditions can be determined by varying one or more of the parameters given as an example, as known in the art, while maintaining a similar degree of identity or similarity between the target nucleic acid molecule and the primer or probe used.

### Vectors/Host Cells

The invention also provides vectors containing the nucleic acid molecules described herein. The term “vector” refers to a vehicle, preferably a nucleic acid molecule, which may transport the nucleic acid molecules. When the vector is a nucleic acid molecule, the nucleic acid molecules are covalently linked to the vector nucleic acid. With this aspect of the invention, the vector includes a plasmid, single or double stranded phage, a single or double stranded RNA or DNA viral vector, or artificial chromosome, such as a BAC, PAC, YAC, OR MAC.

A vector may be maintained in the host cell as an extrachromosomal element where it replicates and produces additional copies of the nucleic acid molecules. Alternatively, the vector may integrate into the host cell genome and produce additional copies of the nucleic acid molecules when the host cell replicates.

The invention provides vectors for the maintenance (cloning vectors) or vectors for expression (expression vectors) of the nucleic acid molecules. The vectors may function in prokaryotic or eukaryotic cells or in both (shuttle vectors).

Expression vectors contain cis-acting regulatory regions that are operably linked in the vector to the nucleic acid molecules such that transcription of the nucleic acid molecules is allowed in a host cell. The nucleic acid molecules may be introduced into the host cell with a separate nucleic acid molecule capable of affecting transcription. Thus, the second nucleic acid molecule may provide a trans-acting factor interacting with the cis-regulatory control region to allow transcription of the nucleic acid molecules from the vector. Alternatively, a trans-acting factor may be supplied by the host cell. Finally, a trans-acting factor may be produced from the vector itself. It is understood, however, that in some embodiments, transcription or translation of the nucleic acid molecules may occur in a cell-free system.

The regulatory sequence to which the nucleic acid molecules described herein may be operably linked include promoters for directing mRNA transcription. These include, but are not limited to, the left promoter from bacteriophage X, the lac, TRP, and TAC promoters from E. coli, the early and late promoters from SV40, the CMV immediate early promoter, the adenovirus early and late promoters, and retrovirus long-terminal repeats.

In addition to control regions that promote transcription, expression vectors may also include regions that modulate transcription, such as repressor binding sites and enhancers. Examples include the SV40 enhancer, the cytomegalovirus immediate early enhancer, polyoma enhancer, adenovirus enhancers, and retrovirus LTR enhancers.

In addition to containing sites for transcription initiation and control, expression vectors may also contain sequences necessary for transcription termination and, in the transcribed region a ribosome binding site for translation. Other regulatory control elements for expression include initiation and termination codons as well as polyadenylation signals. The person of ordinary skill in the art would be aware of the numerous regulatory sequences that are useful in expression vectors. Such regulatory sequences are described, for example, in Sambrook et al., Molecular Cloning: A Laboratory Manual, 3^{rd} ed. (2001) Cold Spring Harbor Laboratory Press, Cold Spring Harbor, N.Y.

A variety of expression vectors may be used to express a nucleic acid molecule. Such vectors include chromosomal, episomal, and virus-derived vectors, for example vectors derived from bacterial plasmids, from bacteriophage, from yeast episomes, from yeast chromosomal elements, including yeast artificial chromosomes, from viruses such as baculoviruses, papovaviruses such as SV40, Vaccinia viruses, adenoviruses, poxviruses, pseudorabies viruses, and retroviruses. Vectors may also be derived from combinations of these sources such as those derived from plasmid and bacteriophage genetic elements, e.g. cosmids and phagemids. Appropriate cloning and expression vectors for prokaryotic and eukaryotic hosts are described in Sambrook et al., ibid.

The regulatory sequence may provide constitutive expression in one or more host cells (i.e. tissue specific) or may provide for inducible expression in one or more cell types such as by temperature, nutrient additive, or exogenous factor such as a hormone or other ligand. A variety of vectors providing for constitutive and inducible expression in prokaryotic and eukaryotic hosts are well known to those of ordinary skill in the art.

The nucleic acid molecules may be inserted into the vector nucleic acid by well-known methodology. Generally, the DNA sequence that will ultimately be expressed is joined to an expression vector by cleaving the DNA sequence and the expression vector with one or more restriction enzymes and then ligating the fragments together. Procedures for restriction enzyme digestion and ligation are well known to those of ordinary skill in the art.

The vector containing the appropriate nucleic acid molecule may be introduced into an appropriate host cell for propagation or expression using well-known techniques. Bacterial cells include, but are not limited to, E. coli, Streptomyces, and Salmonella typhimurium. Eukaryotic cells include, but are not limited to, yeast, insect cells such as Drosophila, animal cells such as COS and CHO cells, and plant cells.

As described herein, it may be desirable to express a peptide of the invention as a fusion protein, especially when expressing a complete reporter fragment pair member construct. Accordingly, the invention provides vectors that allow for the production of such peptides. These vectors may increase the expression of a recombinant protein, increase the solubility of the recombinant protein, and aid in the purification of the protein by acting for example as a ligand for affinity purification. A proteolytic cleavage site may be introduced at the junction of the fusion moiety so that the desired peptide may ultimately be separated from the fusion moiety. Proteolytic enzymes include, but are not limited to, factor Xa, thrombin, and enteroenzyme. Typical fusion expression vectors include pGEX (Smith et al., Gene 67:31-40 (1988)), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein. Examples of suitable inducible non-fusion E. coli expression vectors include pTrc (Amann et al., Gene 69:301-315 (1988)) and pET 11d (Studier et al., Gene Expression Technology: Methods in Enzymology 185:60-89 (1990)).

Recombinant protein expression may be maximized in host bacteria by providing a genetic background wherein the host cell has an impaired capacity to proteolytically cleave the recombinant protein. (Gottesman, S., Gene Expression Technology: Methods in Enzymology 185, Academic Press, San Diego, Calif. (1990)119-128). Alternatively, the sequence of the nucleic acid molecule of interest may be altered to provide preferential codon usage for a specific host cell, for example E. coli. (Wada et al., Nucleic Acids Res. 20:2111-2118 (1992)).

The nucleic acid molecules may also be expressed by expression vectors that are operative in yeast. Examples of vectors for expression in yeast e.g., S. cerevisiae include pYepSec1 (Baldari, et al, EMBO J. 6:229-234 (1987)), pMFa (Kujan et al., Cell 30:933-943 (1982)), pJRY88 (Schultz et al., Gene 54:113-123 (1987)), and pYES2 (Invitrogen Corporation, San Diego, Calif.).

The nucleic acid molecules may also be expressed in insect cells using, for example, baculovirus expression vectors. Baculovirus vectors available for expression of proteins in cultured insect cells (e.g., Sf 9 cells) include the pAc series (Smith et al., Mol. Cell. Biol. 3:2156-2165 (1983)) and the pVL series (Lucklow et al., Virology 170:31-39 (1989)).

In certain embodiments of the invention, the nucleic acid molecules described herein are expressed in mammalian cells using mammalian expression vectors. Examples of mammalian expression vectors include pCDM8 (Seed, B. Nature 329:840 (1987)) and pMT2PC (Kaufman et al., EMBO J. 6:187-195 (1987)).

The expression vectors listed herein are provided by way of example only of the well-known vectors available to those of ordinary skill in the art that would be useful to express the nucleic acid molecules. The person of ordinary skill in the art would be aware of other vectors suitable for maintenance propagation or expression of the nucleic acid molecules described herein. These are found for example in Sambrook et al., ibid.

The invention also encompasses vectors in which the nucleotide sequences described herein are cloned into the vector in reverse orientation, but operably linked to a regulatory sequence that permits transcription of antisense RNA. Thus, an antisense transcript may be produced to all, or to a portion, of the nucleic acid molecule sequences described herein, including both coding and non-coding regions. Expression of this antisense RNA is subject to each of the parameters described above in relation to expression of the sense RNA (regulatory sequences, constitutive or inducible expression, tissue-specific expression).

The invention also relates to recombinant host cells containing the vectors described herein. Host cells therefore include prokaryotic cells, lower eukaryotic cells such as yeast, other eukaryotic cells such as insect cells, and higher eukaryotic cells such as mammalian cells. Host cells can include, but are not limited to, silkworm larvae, CHO cells, E. coli, and yeast.

The recombinant host cells are prepared by introducing the vector constructs described herein into the cells by techniques readily available to the person of ordinary skill in the art. These include, but are not limited to, calcium phosphate transfection, DEAE-dextran-mediated transfection, cationic lipid-mediated transfection, electroporation, transduction, infection, lipofection, and other techniques such as those found in Sambrook, et al., ibid.

Host cells may contain more than one vector. Thus, different nucleotide sequences may be introduced on different vectors of the same cell. Similarly, the nucleic acid molecules may be introduced either alone or with other nucleic acid molecules that are not related to the nucleic acid molecules such as those providing trans-acting factors for expression vectors. When more than one vector is introduced into a cell, the vectors may be introduced independently, co-introduced or joined to the nucleic acid molecule vector.

In the case of bacteriophage and viral vectors, these may be introduced into cells as packaged or encapsulated virus by standard procedures for infection and transduction. Viral vectors may be replication-competent or replication-defective. In the case in which viral replication is defective, replication will occur in host cells providing functions that complement the defects.

Vectors generally include selectable markers that enable the selection of the subpopulation of cells that contain the recombinant vector constructs. The marker may be contained in the same vector that contains the nucleic acid molecules described herein or may be on a separate vector. Markers include tetracycline or ampicillin-resistance genes for prokaryotic host cells and dihydrofolate reductase or neomycin resistance for eukaryotic host cells. However, any marker that provides selection for a phenotypic trait will be effective.

While the mature proteins may be produced in bacteria, yeast, mammalian cells, and other cells under the control of the appropriate regulatory sequences, cell-free transcription and translation systems may also be used to produce these proteins using RNA derived from the DNA constructs described herein.

Where secretion of the peptide is desired, which is difficult to achieve with multi-transmembrane domain containing proteins such as enzymes, appropriate secretion signals are incorporated into the vector. The signal sequence may be endogenous to the peptides or heterologous to these peptides.

Where the peptide is not secreted into the medium, the protein may be isolated from the host cell by standard disruption procedures, including freeze thaw, sonication, mechanical disruption, use of lysing agents and the like. The peptide may then be recovered and purified by well-known purification methods including ammonium sulfate precipitation, acid extraction, anion or cationic exchange chromatography, phosphocellulose chromatography, hydrophobic-interaction chromatography, affinity chromatography, gel filtration, hydroxylapatite chromatography, lectin chromatography, or high performance liquid chromatography.

In one embodiment, the polypeptide is expressed in a bacterial host cell, such as E. coli, and the protein extracted from inclusion bodies present in the host cell using chaotropic agents such as guanidium hydrochloride (GuHCl) or urea to solubilise the protein. The polypeptide is then bound to a solid phase, such as a chromatography matrix, via a fusion tag present on the polypeptide, e.g. a 6×His tag. Once the solid phase has been washed to remove contaminants, the polypeptide is preferably refolded whilst still bound to the solid phase by applying a concentration gradient of the chaotropic agent (as the concentration of the agent decreases, the polypeptide refolds). We have found that using this methodology results in reporter fragments having good in vitro activity in homogenous assays.

It is also understood that depending upon the host cell in recombinant production of the peptides described herein, the peptides may have various glycosylation patterns, depending upon the cell, or maybe non-glycosylated as when produced in bacteria. In addition, the peptides may include an initial modified methionine in some cases as a result of a host-mediated process.

The recombinant host cells expressing the peptides described herein have a variety of uses. First, the cells are useful for producing an enzyme protein or peptide that may be further purified to produce desired amounts of enzyme protein or fragments. Thus, host cells containing expression vectors are useful for peptide production.

Host cells are also useful for conducting cell-based assays involving the enzyme protein or enzyme protein fragments, such as those described above as well as other formats known in the art. Thus, a recombinant host cell expressing a native enzyme protein is useful for assaying compounds that stimulate or inhibit enzyme protein function.

Host cells are also useful for identifying enzyme protein mutants in which these functions are affected. If the mutants naturally occur and give rise to a pathology, host cells containing the mutations are useful to assay compounds that have a desired effect on the mutant enzyme protein (for example, stimulating or inhibiting function) which may not be indicated by their effect on the native enzyme protein.

### Homogeneous Assays

The reporter fragments, polypeptides and reporter components of the present invention can be used in in vitro assays to determine the presence of a target analyte of interest. Analytes of interest include those present in environment or biological samples. Biological samples include whole blood, serum, saliva and urine. The term “in vitro” is taken to mean in the present context that the assays are conducted outside of living cells, such as in cell-free assays.

The assays methods of the invention typically comprise mixing a sample with reporter components and determining the presence or absence (or the extent of) a detectable signal resulting from an association of reporter fragments/report components mediated by binding of the reporter components to the analyte of interest.

The detectable signal may, for example, be a colorimetric signal, a fluorescent signal or a chemiluminescent signal. By way of example, beta-lactamase can cleave nitrocefin to produce a colour change from yellow to red. Luciferases act on the substrate luciferin to generate a chemiluminescent signal.

The amount of reporter polypeptide required for an in vitro assay is generally higher than that found in vivo. For example, the concentration of report polypeptide in the reaction mix may be at least 1 μM, such as at least 10 or 100 μM, or 1 nM. Accordingly, it may be desirable to include agents in the reaction mix that reduce the possibility of spontaneous association between members of a reporter complex. Such agents include chaotropic agents/protein denaturants, such as urea (e.g. at a concentration of from 200 to 700 mM, such as about 500 mM). It was determined in example 1 that at a low concentration of urea the enzyme fragments were partially denatured, thereby reducing their natural affinity for each other (spontaneous complementation) whilst still allowing for interaction and conformational change when directed into close proximity by the analyte (forced complementation).

Other agents that it may also be desirable to include to assist in reducing background/increasing the signal to noise ratio are: (i) solvents such as ethanol (preferably from 2% to 10% v/v, such as from 3% to 8% v/v); isoproponal (preferably from 2% to 10% v/v, such as from 3% to 8% v/v); methanol (preferably from 5% to 10% v/v); DMSO (preferably from 10% to 20% v/v) or acetonitrile (preferably from 2% to 10% v/v, such as from 3% to 8% v/v); (ii) BSA; (iii) detergents such as Tween-20 (e.g. from 0.1% to 0.25% v/v or Triton-X100 (preferably greater than 0.2%, such as from 0.2% to 0.4% v/v); (iv) salts e.g. NaCl, K₂SO₄ or (NH₄)₂SO₄; and/or (v) imidazole (preferably from 5 to 20 mM, such as from 5 to 15 mM).

The reporter polypeptides and reporter components of the invention are not limited to use in in vitro homogenous assays. They may also be used in in vitro heterogeneous assays where at least one reporter fragment or reporter component is immobilized to a solid phase. They may also be used in in vivo assays, with the components typically being introduced into cells by transforming/transfecting cells with nucleotide constructs of the invention which are capable of directing the expressing of the reporter polypeptides in the cells under suitable conditions.

### Kits

The present invention also provides reporter fragments and/or reporter components as kits. Such kits may be used for FEC assays, such as in vitro assays for determining the presence and/or amount of an analyte in a sample. Kits comprises one or more reporter polypeptide fragments and/or reporter components of the invention. The kits typically comprise a plurality of polypeptide fragments and/or reporter components of the invention, such as a pair, which together can form an active polypeptide complex capable of generating a detectable signal in the presence of an analyte of interest.

Kits may also include instructions for use. Other optional components include buffers, standards, detection reagents and the like.

### EXAMPLES

The following examples are included to demonstrate preferred embodiments of the invention. It should be appreciated by those of skill in the art that the techniques disclosed in the examples that follow represent techniques discovered by the inventor to function well in the practice of the invention, and thus may be considered to constitute preferred modes for its practice. However, those of skill in the art should, in light of the present disclosure, appreciate that many changes may be made in the specific embodiments which are disclosed and still obtain a like or similar result without departing from the spirit and scope of the invention.

### Example 1

### Homogeneous In Vitro FEC Assays for Divalent Cations

The procedures outlined herein describe the synthesis and characterisation of TEM1 fragments generated by splitting the full-length parental enzyme at amino acids 196/197 followed by introduction of a flexible linker (G₄S) and histidine tag (6×H) at the break-point termini. The subsequent α fragment (PB11) and ω fragment (PB13) were used to introduce point mutations in order to reduce hydrophobic interactions and aggregation and increase protein stability. These changes to the amino acid sequences of the fragments resulted in the generation of the αV74TM182T fragment (PB11.2) and the ωM211Q fragment (PB13.1).

These peptides and nucleic acids encoding them were used to construct reporter fragment pair members comprising flexible linkers (G₄S) and interactor domains (polyhistidine tags (6×H)). DNA sequence data indicating the locations of the flexible linker (G₄S), polyhistidine tags (6×His), and point mutations is provided. These fragment pair members were isolated and purified and used to constitute an operable homogeneous in vitro FEC assay for the presence of an analyte.

### PCR of TEM1 Gene Encoding α and ω Fragments

The bia gene encoding TEM1 β lactamase was amplified from pUC18 using the polymerase chain reaction (PCR). All amplifications were performed using Platinum Pfx DNA polymerase (Invitrogen cat. 11708-021) according to the manufacturer's recommendations. Custom-made oligonucleotide primers were purchased from SigmaGenosys (Australia). The α fragment of TEM1 with a C-terminal G₄S linker and histidine tag was amplified using forward primer FEC16 and reverse primer FEC27. The ω fragment of TEM1 with an N-terminal histidine tag and G₄S linker was amplified using forward primer FEC24 and reverse primer FEC29 (see Table 1—Primer Sequences for a description of the primers used for PCR, sequencing and mutagenesis). For cloning of PCR products into pET-26b(+), the destination vector, an NdeI restriction site was incorporated at the 5′ end of the forward primers and a XhoI restriction site was incorporated at the 5′ end of the reverse primers.

A 4 μl aliquot of each PCR reaction was analysed on a 1% TAE agarose gel (100V for 30 min).

TABLE 1
Primer Sequences

| Oligo | Full Name | Sequence (5′ - 3′) | SEQ ID NO: |
|---|---|---|---|
| FEC10 | pET-T7terminator seq | GCTAGTTATTGCTCAGCGG | 25 |
| FEC11 | pET-T7promoter seq | TAATACGACTCACTATAGGGG | 26 |
| FEC16 | TEM1newNdeIBL26F1 | CTTTTCCCTGTCCATATGCACCCAGAAACGCTG | 27 |
| | | GTGAAAG | |
| FEC24 | FEC24:NdeI-6H-G4S- | CTTTTCCCTGTCCATATGCATCACCATCATCAT | 28 |
| | TEM1-BL197-F1 | CACGGTGGAGGTGGCTCGGAACTACTTACTCT | |
| | | AGCTTCCCGGCAAC | |
| FEC27 | FEC27:XhoI-stop-6H- | AACCGGTATTCCCTCGAGTCAGTGATGATGATG | 29 |
| | G4S-TEM1-BL196-R1 | GTGATGCGAGCCACCTCCACCGCCAGTTAATA | |
| | | GTTTGCGCAACGTTGTTGC | |
| FEC29 | FEC29:XhoI-stop-TEM1- | AACCGGTATTCCCTCGAGTCACCAATGCTTAAT | 30 |
| | BL290-R1 | CAGTGAGGCACC | |
| FEC47 | BL-V74T-F1 | GCACTTTTAAAACGCTGCTATGTGGC | 31 |
| FEC48 | BL-V74T-R1 | GCCACATAGCAGCGTTTTAAAAGTGC | 32 |
| FEC49 | BL-M211Q-F1 | CAATTAATAGACTGGCAGGAGGCGGATAAAG | 33 |
| FEC50 | BL-M211Q-R1 | CTTTATCCGCCTCCTGCCAGTCTATTAATTG | 34 |
| FEC55 | BL-M182T-F1 | GTGACACCACGACACCTGTAGCAATGGCAAC | 35 |
| FEC56 | BL-M182T-R1 | GTTGCCATTGCTACAGGTGTCGTGGTGTCAC | 36 |
| FEC67 | PB11LongLinker-F | CTATTAACTGGCGGTGGAGGTGGAAGCGGCG | 37 |
| | | GAGGTGGTTCTGGTGGTGGA | |
| FEC68 | PB11LongLinker-R | GTCAGTGATGATGATGGTGATGCGAGCCTCCA | 38 |
| | | CCACCAGAACCACCTCCGCCGCTTCC | |
| FEC75 | PB13LLF | GGTGGTGGAGGCAGCGGCGGAGGTGGTTCTG | 39 |
| | | GTGGAGGTGGCTCGGAACTAC | |
| FEC76 | PB13LLR | AGAACCACCTCCGCCGCTGCCTCCACCACCGT | 40 |
| | | GATGATGATGGTGATGCATATG | |

Purification of PCR Products and Cloning into E. coli Host Cells

PCR products were purified directly from the reaction tube using the QIAquick PCR purification kit (Qiagen cat. 28104) according to the manufacturer's instructions.

PCR products were cloned into the NdeI/XhoI site of the pET-26b(+) vector (Novagen cat. 69862-3), a prokaryotic expression vector that allows for inducible expression of recombinant proteins in E. coli. Ligated plasmids were transformed into BL21-Gold (DE3) competent cells (Stratagene cat 230132) according to the manufacturer's instructions. Five to ten colonies from each transformation were screened to confirm the presence of the cloned insert sequence within the pET-26b(+) vector by restriction digestion with NdeI/XhoI as previously described and analysis of digestion products by gel electrophoresis. Particular clones were also checked by DNA sequencing (using primers FEC10 and FEC11—Table 1) to confirm the mutations made.

### Site-Directed Mutagenesis

Site-directed mutagenesis of α fragment (PB11) and ω fragment (PB13) was done using the QuickChange II XL-Site-Directed Mutagenesis Kit (Stratagene cat.200522) as described in the manufacturer's instruction manual (Rev# 124001e). Sequence manipulation and primer design was done using Vector NTI 9.0.0 (Sep. 2, 2003) taking into consideration codon usage frequencies of E. coli (accessible at www.kazusa.or.jp/codon). Primers (refer to Table 1 for a detailed description of the forward and reverse primers used) were synthesised and purified using HPLC by SigmaGenosys (Australia).

Briefly, α fragment and ω fragment plasmid DNA isolated and quantitated as previously described was used as the template in PCR amplified mutagenesis reactions as follows; 25 ng template DNA was added to 125 ng forward primer (FEC47 or FEC49), 125 ng reverse primer (FEC48 or FEC50), 1× Reaction Buffer, 1 μl dNTP mix, 3 μl QuickSolution and ultra pure water to a final volume of 50 μl. 1 μl PfuUltra HF DNA polymerase (2.5 U/μl) was then added and temperature cycling was performed with a Mastercycler ep gradient thermal cycler (Eppendorf). For introduction of the V74T and M211Q point mutations PCR cycling was done as follows; denaturation at 95° C.×1 min, and 18 cycles of 95° C.×50 sec (denaturation), 60° C.×50 sec (annealing) and 68° C.×6 min (extension) with a final extension step of 68° C.×6 min at cycling completion. The PCR cycling conditions for introduction of the M182T mutation varied slightly with annealing done at 65° C.×50 sec using αV74T fragment as template and FEC55 and FEC56 forward and reverse primers respectively. PCR amplified mutagenesis reactions were subsequently digested with 1 μl Dpn I restriction enzyme (10 U/μl) added directly to each reaction and incubated at 37° C. for 1 hour to digest parental non-mutated DNA. Two μl of Dpn I treated DNA from each sample reaction was then used to transform XL10-Gold Ultracompetent Cells as outlined in the QuickChange II XL-Site-Directed Mutagenesis Kit manufacturer's instruction manual (Rev# 124001e). E. coli transformants were screened as outlined previously, with resultant plasmid DNA sequenced to confirm correct insertion of point mutations. Plasmid DNA incorporating desired mutations was then used to transform BL21-Gold (DE3) competent cells as described for protein expression and purification.

The introduction of these point mutations resulted in increased fragment solubility and decreased aggregation of the final product, which are significant advantages for use in homogeneous in vitro FEC assays as well as process manufacturing (e.g. Table 2).

TABLE 2

| | | Purification | FPLC | Purification |
|---|---|---|---|---|
| Protein | Fragment | (solubility) | (aggregation) | Strategy |
| PB11 | α | + | ++ | Native Ni-NTA |
| PB11.2 | α | ++ | + | |
| PB13 | ω | ++ | +++ | |
| PB13.1 | ω | +++ | + | |
| PB15 | FL | <+ | N/A | |
| PB15.1 | FL | + | +++ | |
| PB15.3 | FL | ++ | N/A | |
| PB11 | α | ++ | + | Denaturing |
| | | | | CRF Ni-NTA |
| PB11.2 | α | +++ | <+ | |
| PB13 | ω | +++ | ++ | |
| PB13.1 | ω | ++ | + | |
| PB15 | FL | >+++ | + | |
| PB15.1 | FL | >+++ | + | |
| PB15.3 | FL | >+++ | + | |

### Purification and Characterisation of α and ω Fragments

All four enzyme fragments [α fragment (PB11), ω fragment (PB13), αV74TM182T fragment (PB112) and ωM211Q fragment (PB13.1)] were expressed, purified and characterised in the same manner, in order to directly compare their rate of catalysis and signal to noise ratio upon forced enzyme complementation with Ni²⁺ as described below.

### Expression of α Fragment (PB11) and ω Fragment (PB13)

Expression of fragments in BL21-Gold (DE3) was done as follows; 10 ml of LB broth supplemented with 50 μg/ml of kanamycin was inoculated with a single colony of interest and the liquid culture was incubated overnight (−14 hours) at 37° C. with shaking at 250 rpm. This culture was then used to inoculate 250 ml Overnight Express Instant TB Medium (Novagen cat. 71491-4) supplemented with 50 μg/ml of kanamycin in a 2 L conical flask. The cultures were incubated for 24 hours at 37° C. with shaking at 250 rpm.overnight. The Overnight Express Instant TB Medium used here allows for auto-induction of protein expression at a high bacterial density (refer to Novagen User Protocol TB383 Rev. F 0505). Optimal protein expression occurs when the bacterial culture has reached the stationary phase, which was determined to occur within 24 hours based on optical density readings taken at 600 nm. Cells were subsequently pelleted at 3,220×g for 30 min (4° C.) and the supernatants were discarded. Cell pellets were stored at −20° C. until protein purification.

### Extraction of Recombinant Proteins Under Denaturing Conditions

The pellet from a 250 ml overnight induction was lysed in 25 ml of [6 M GuHCl-100 mM NaH₂PO₄ 10 mM Tris pH 8] followed by a 1-hour incubation at 4° C. with shaking at 100 rpm. To enhance lysis, the suspension was sonicated in an ice bath for 5 cycles of 30 seconds on/30 seconds off using a Branson 250 sonifier. After sonication, the lysate was centrifuged at 12,000×g for 30 min (4° C.) and then passed through a 0.2 μm filter to remove cellular debris.

### Immobilised Metal Affinity Chromatography (IMAC) and On-Column Refolding

Recombinant His-tagged proteins were purified using a 1 ml HisTrap HP column (Amersham cat. 17-5247-01) under the control of a DuoFlow chromatography system (BioRad cat. 760-0037). The HisTrap column was equilibrated with 10 column volumes (CV) of 8 M urea 100 mM NaH₂PO₄ 10 mM Tris pH 7.5 at a flow rate of 1 ml/min. Cleared E. coli lysates were then loaded onto the column using a Model EP-1 Econopump (BioRad cat. 731-8142) at 0.5 ml/min. The column was washed with 10 CV of 8 M Urea 100 mM NaH₂PO₄ 10 mM Tris pH 6.3. Bound protein was refolded over a 60 CV gradient from 8 M Urea 100 mM NaH₂PO₄ 10 mM Tris 200 mM L-arginine 100 μM GSSG pH 7.5 to 100 mM NaH₂PO₄ 10 mM Tris 200 mM L-arginine 100 μM GSSG pH 7.5 at 1 ml/min. Histidine-tagged proteins were eluted with 10 CV of 250 mM imidazole 50 mM NaH₂PO₄ 150 mM NaCl pH 8, and analysed by PAGE, western blotting, gel filtration and mass spectroscopy.

On-column refolding of denatured fragments was found to be very effective in the production of both the α and ω fragments of beta-Lactamase (wild-type and mutated). This method carriers advantages over the purification method of the native material and is suitable for large-scale process manufacturing.

### Gel Filtration

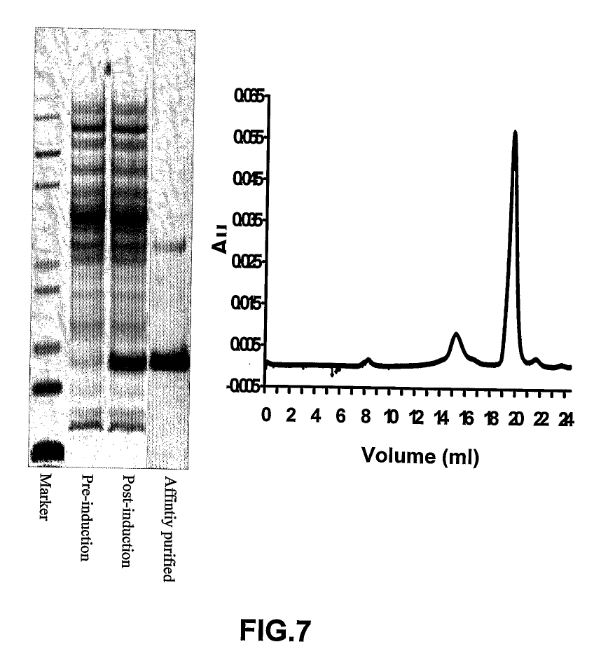

In some cases, following affinity purification of the enzyme fragments, pooled eluates were subjected to size exclusion chromatography using a Superdex200GL column (Amersham cat. 17-5175-01) under the control of a DuoFlow chromatography system (BioRad cat. 760-0037). Gel filtration was used for either polishing of purified enzyme fragments (removal of contaminating proteins) or to determine the proportion of monomeric versus aggregated enzyme fragments after on-column refolding. The protocol was as follows: equilibration with 2 CV of 50 mM NaH₂PO₄ 150 mM NaCl, pH7 at 0.6 ml/min; injection of 250-500 μl of sample at 0.6 ml/min; and isocratic flow of 1 CV of 50 mM NaH₂PO₄ 150 mM NaCl, pH 7 at 0.6 ml/min. See FIG. 7 for exemplary results.

### Mass Spectroscopy

The molecular weight of gel-filtration purified α and ω fragments were determined using the HPLC/TOF mass spectroscopy service at the Institute for Molecular Bioscience, University of Queensland, Australia. Respective experimental data was collected and analysed for comparison with the theoretical molecular weight values.

### Enzyme Fragment Complementation

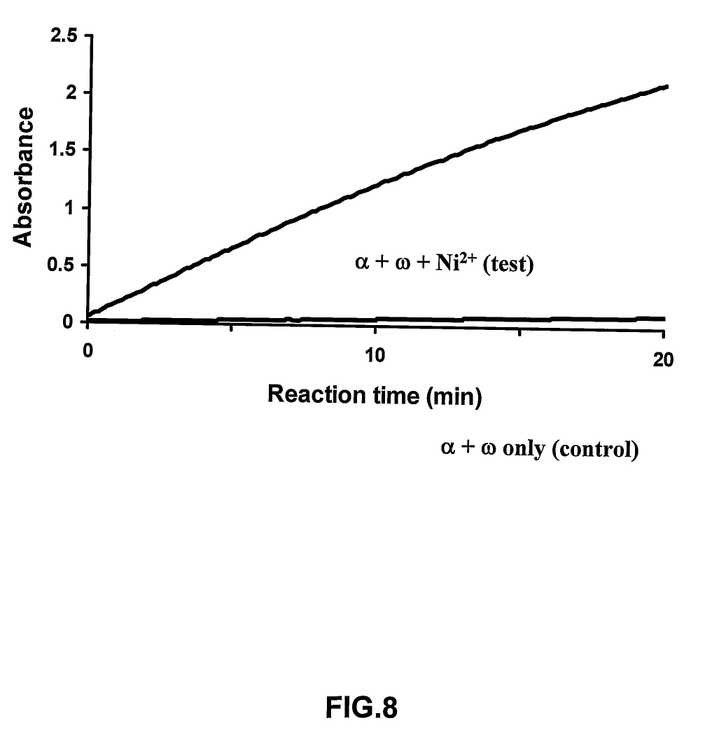

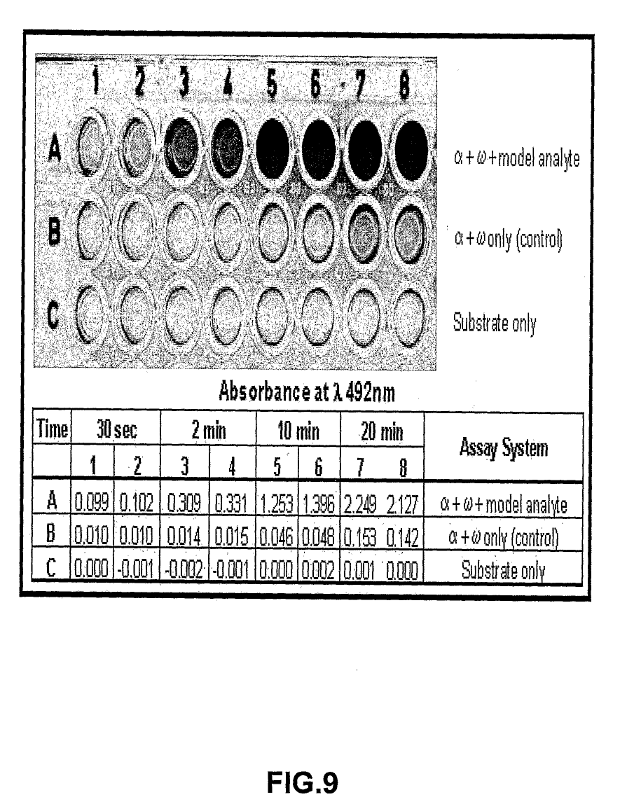

Purified α and ω fragments containing the 6-histidine-tag were used for kinetic studies to calculate the signal (Ni²⁺ forced enzyme complementation) to noise (spontaneous enzyme complementation without Ni²⁺) ratio. Different substrates, buffer additives and inhibitors were used to investigate and evaluate the signal to noise ratio of forced enzyme complementation. Exemplary results in one embodiment employing equimolar amounts of α and ω fragments of the invention are provided in FIG. 8 and FIG. 9.

### Nitrocefin Activity Assays

Nitrocefin (Merck, Australia) is a colorimetric substrate of TEM-1 beta-lactamase that changes colour from yellow to red (OD₄₉₂) following hydrolysis of the β-lactam ring. Hence, nitrocefin assays were performed to assess the enzymatic activity of enzyme fragment complementation. Assays were performed using a 96-well flat bottom cell-culture plate (TPP, Australia). Nitrocefin stock solutions of 200 or 600 μM were prepared in 50 mM NaH₂PO₄; 150 mM NaCl; 5% DMSO (Sigma, Australia) pH 7. For each 200 μl reaction, 100 μl of the nitrocefin stock was added to 100 μl of enzyme fragments (with or without Ni²⁺) in 50 mM NaH₂PO₄ 150 mM NaCl pH 7. Depending on the final signal required, the concentration of enzyme fragments used per well ranged from 40 nM to 200 nM and the final concentration of the Ni²⁺ analyte from 100 μM or 200 μM. Assay components were mixed well by pipetting, incubated for 5 minutes at room temperature. Kinetics of the nitrocefin hydrolysis were read at 492 nm using a SpectraMax190 (Molecular Devices, USA).

### Effect of Various Buffer Additives

In an attempt to improve the signal to noise ratio of forced α and ω fragments complementation, the effect of different buffer additives such as solvents, BSA, detergents, protein denaturants and salts were investigated. The stop solution Tazobactam was also investigated. Assays were performed as described herein.

### Solvents

As a low concentration of solvent could partially denature the enzyme fragments, it was investigated whether a small amount of ethanol, isopropanol, methanol, DMSO and acetonitrile would reduce natural fragment complementation and increase the signal to noise ratio. The final concentrations of each solvent tested were 15%, 7.5%, 3.75%, 1.875%, 0.9875% and 0%. Kinetics of the complementation assays was read at 492 nm for 30 minutes. The signal/noise ratio was then calculated according to the maximum rate (mOD/min) from SoftmaxPro. It was determined that an optimal signal to noise ratio was achieved with ethanol, isoproponal, methanol, DMSO and acetonitrile at concentrations of 3.75%, 3.75%, 7.5%, 15% and 3.75% respectively. For example, the effect of ethanol in final concentration range from 15%, 7.5%, 3.75%, 1.875% and 0%, the signal/noise ratio was 7.1, 8.4, 8.5, 5.4 and 3.7 respectively.

It was determined that at certain range of concentration of solvent the enzyme fragments were partially denatured, thereby reducing their natural affinity for each other (spontaneous complementation) whilst still allowing for interaction and conformational change when directed into close proximity by the Ni²⁺ analyte (forced complementation).

### BSA

BSA (Pierce, USA) is widely used as the protein stabiliser; using BSA might help preventing the natural fragment complementation by interfering the close contact of two fragments to increase the signal/noise ratio. Final concentration range (mg/ml) was 0.6, 0.3, 0.15, 0.075, 0.0375, 0.01875 and 0. Reading data collecting time was 30 minutes. The signal/noise ratio was then calculated according to the maximum rate (mOD/min) from SoftmaxPro. Adding BSA increases both signal and background, so the final signal/noise ratio did not change significantly, but the signal output was amplified nearly 2 fold. It was determined that the BSA would provide the crowding effect in the assay solution to amplify both signal and background.

### Detergents

Detergent is widely used to reduce protein-protein interaction or ELISA background; using small amount might help preventing the natural fragment complementation by keeping two fragments apart then increased the signal/noise ratio. Tween-20 and Triton-X100 were used and the final concentration range was 0.3%, 0.15%, 0.075%, 0.0375%, 0.01875% and 0%. Reading data collecting time was 30 minutes. The signal/noise ratio was then calculated according to the maximum rate (mOD/min) from SoftmaxPro.

It was determined that an optimal signal to noise ratio was achieved with Tween-20 and Triton-X100 at concentrations of 0.15% and 0.3% respectively. For example, the effect of TX-100 tested in final concentration range from 0.3%, 0.15%, 0.075%, 0.0375%, 0.01875% and 0%, the signal/noise ratio was 14.1, 6.9, 3.8, 2.7, 1.8, and 3.9 respectively. Note that the activity decreases when increasing detergent concentrations.

It was determined that at certain range of concentration of detergent, the enzyme fragments were partially denatured, thereby reducing their natural affinity for each other (spontaneous complementation) whilst still allowing for interaction and conformational change when directed into close proximity by the Ni²⁺ analyte (forced complementation).

### Denaturants

To test the effect of protein denaturants on enzyme fragment complementation, nitrocefin activity assays were performed using a matrix of fragment, substrate and NiSO₄ concentrations in the presence of urea or guanidine hydrochloride. In preliminary assays, the complementation of 40 nM fragments was tested in concentrations of urea ranging from 2 M to 62.5 mM and in concentrations of GuHCl that ranged from 1M to 31.25 mM. The assay was prepared as follows: 40 nM of each fragment was added to a two-fold dilution series of urea [from 2 M urea 50 mM NaH₂PO₄ 150 mM NaCl pH7 to 62.5 mM urea 50 mM NaH₂PO₄ 150 mM NaCl pH7] or guanidine hydrochloride [from 1 M GuHCl 50 mM NaH₂PO₄ 150 mM NaCl pH7 to 31.25 mM GuHCl 50 mM NaH₂PO₄ 150 mM NaCl pH7] in duplicate. To the first set of wells, NiSO₄ was added to a final concentration of 100 μM, whereas the second set contained no NiSO₄. The plate was incubated for 5 minutes and the kinetics read at 492 nm using a SpectroMax reader. It was determined that an optimal signal to noise ratio was achieved with urea at a concentration of 0.5 M and in subsequent experiments the amount of fragments was increased up to 200 nM and the NiSO₄ and substrate concentration up to 200 μM and 300 μM respectively to achieve an the largest possible signal with a low background.

It was determined that at a low concentration of urea the enzyme fragments were partially denatured, thereby reducing their natural affinity for each other (spontaneous complementation) whilst still allowing for interaction and conformational change when directed into close proximity by the Ni²⁺ analyte (forced complementation).

### Salts

Electrostatic effects have an important function in both enzyme catalysis and protein-protein interactions, and these effects can be modulated or modified using different salts. Three different salts were tested for their effect on forced enzyme complementation: NaCl, K₂SO₄ and (NH₄)₂SO₄. The assay was prepared as follows: 40 nM of each fragment was added to a two-fold dilution series of NaCl [from 2 M NaCl 50 mM NaH₂PO₄ pH 7 to 62.5 mM NaCl 50 mM NaH₂PO₄ pH7], K₂SO₄ [from 0.4 M K₂SO₄ 50 mM NaH₂PO₄ pH 7 to 25 mM K₂SO₄ 50 mM NaH₂PO₄ pH 7] or (NH₄)₂SO₄ [from 0.4 M (NH₄)₂SO₄ 50 mM NaH₂PO₄ pH 7 to 50 mM (NH₄)₂SO₄ 50 mM NaH₂PO₄ pH7] in duplicate. To the first set of wells, 100 μM of NiSO₄ was added and mixed, whereas the second set contained no NiSO₄. The plate was incubated for 5 minutes and the kinetics read at 492 nm using a SpectroMax 190 reader.

Adding salt to the FEC assays increases both signal and background, so the final signal/noise ratio was not change significant, but the signal output was amplified.

### Imidazole

A small amount (20 mM) Imidazole (ICN, Australia) was used as the wash buffer in the histidine-tagged protein purification (www.qiagen.com), so imidazole might prevent the natural fragment complementation by interfering the fragment surface histidines interaction then help to raise the signal/noise ratio. Imidazole final concentration range was 100 mM, 50 mM, 25 mM, 12.5 mM and 0 mM. Reading data collecting time was 30 minutes. The signal/noise ratio was then calculated according to the maximum rate (mOD/min) from SoftmaxPro.

It was determined that an optimal signal to noise ratio was achieved with imidazole at concentrations of 12.5 mM respectively. For example the effect of imidazole in final concentration range from 100 mM, 50 mM, 25 mM, 12.5 mM, 6.25 mM and 0 mM, the signal/noise ratio was 0.8, 2.1, 3.7, 5.3, 5.2, and 4.0 respectively. Note that the activity decreases when increasing imidazole concentrations.

It was determined that, at certain range of concentration of imidazole, the enzyme fragments reduced their natural affinity for each other (spontaneous complementation) whilst still allowing for interaction and conformational change when directed into close proximity by the Ni²⁺ analyte (forced complementation).

### Stop Solution (Tazobactam)

Tazobactam (Sigma, Australia) is one of the most effective inhibitor for TEM-1 beta-lactamase, with reported IC₅₀ 20-50 nM. The IC₅₀ of Tazobactam was determined in-house on full length and complemented fragments at a concentration of 50 nM. So, Tazobactam final concentration between 25 to 50 μM (500-1000 fold more than IC₅₀) should be enough to stop the reaction immediately without changing the OD₄₉₂ value. Tazobactam 25 or 50 μM was used to test the effectiveness of inhibition and continuous monitoring of OD₄₉₂ value after adding Tazobactam was performed for 0.5 to 3 hours. OD₄₉₂ changes obtained were compared and evaluated.

### Example 2

### Homogeneous In Vitro FEC Assays for Anti-Histidine Monoclonal Antibodies

The procedures outlined here describe the synthesis and characterisation of α fragment (PB11.4) and ω fragment (PB13.2) incorporating long flexible linkers [(G₄S)₃] and a histidine tag (6×H) at the break-point termini. DNA sequence data confirmed the presence of the long linkers [(G₄S)₃] and histidine tag (6×H). This fragment pair was used to demonstrate forced enzyme complementation with an antibody (penta-histidine monoclonal antibody binding to histidine tag) in a homogeneous in vitro format assay.

### Site-Directed Mutagenesis

Site-directed mutagenesis by PCR of α (PB11) and ω (PB13) fragments were performed to introduce an additional 10 amino acid linker [(G₄S)₂] into the existing constructs (already containing a 5 amino acid flexible linker (G₄S) at their break-point termini) to produce linkers of 15 amino acids in length (PCR primer sequences are given in Table 1).

Briefly, to generate an α fragment with a long linker (PB 11.4), 25 ng of template DNA was added to 125 ng forward primer (FEC67), 125 ng reverse primer (FEC68), 1× Reaction Buffer, 1 μl dNTP mix, 3 μl QuickSolution and ultra pure water to a final volume of 50 μl. One μl PfuUltra HF DNA polymerase (2.5 U/μl) was then added and temperature cycling was performed with a Mastercycler ep gradient thermal cycler (Eppendorf). PCR cycling was done as follows: denaturation at 95° C.×1 min, and 25 cycles of 95° C.×50 sec (denaturation), 60° C.×30 sec (annealing), 65° C.×30 sec (annealing) and 68° C.×10 min (extension) with a final extension step of 68° C.×7 min at cycling completion.

To generate an α fragment with a long linker (PB13.2), 50 ng of template DNA was added to 125 ng forward primer (FEC75), 125 ng reverse primer (FEC76), 1× Reaction Buffer, 1 μl dNTP mix, 3 μl QuickSolution and ultra pure water to a final volume of 50 μl. One μl PfuUltra HF DNA polymerase (2.5 U/μl) was then added and PCR cycling was done as follows: denaturation at 95° C.×1 min, and 25 cycles of 95° C.×50 sec (denaturation), 60° C.×30 sec (annealing), 65° C.×15 sec (annealing) and 68° C.×10 min (extension) with a final extension step of 68° C.×7 min at cycling completion.

The PCR amplified mutagenesis reactions were digested with 1 μl DpnI as described above. DpnI treated PB11.4 DNA and DpnI treated PB13.2 DNA was used to transform BL21-Gold (DE3) competent cells (Stratagene cat. 230132) according to the manufacturer's instructions. E. coli transformants were screened as outlined previously, with resultant plasmid DNA sequenced (using primers FEC10 and FEC11—Table 1) to confirm correct insertion of long linkers.

### Enzyme Fragment Complementation

Enzyme fragments were expressed, purified and characterised as outlined above. Purified α and ω fragments containing the 15 amino acid long flexible linker and 6-histidine-tag that can bind penta-histidine monoclonal antibody (Ab) were used for kinetic studies to calculate the signal (Ab forced enzyme complementation) to noise (spontaneous enzyme complementation without Ab) ratio.

Nitrocefin hydrolysis was used to measure the enzymatic activity of the Ab forced FEC assay. Assays were performed using a 96-well flat bottom cell-culture plate (TPP, Australia) and nitrocefin hydrolysis was read at 492 nm using a SpectraMax190 (Molecular Devices, USA) plate reader. Briefly, 20 nM of α (PB11.4) and ω (PB13.2) fragments and 33 nM His-Tag monoclonal antibody (Novagen cat.70796-3) were diluted in 0.5 M urea, 0.625 mM Tris, 12.5 mM L-arginine, 6.25 μM oxidised glutathione (GSSG), 50 mM NaH₂PO₄, 150 mM NaCl, pH 7 in a reaction volume of 200 μl and incubated at room temperature for 20 min. Nitrocefin (2 μl of 10 mg/ml in DMSO) was then added to the reaction solution and monitored at 492 nm using a plate reader over a 1 hr time period.

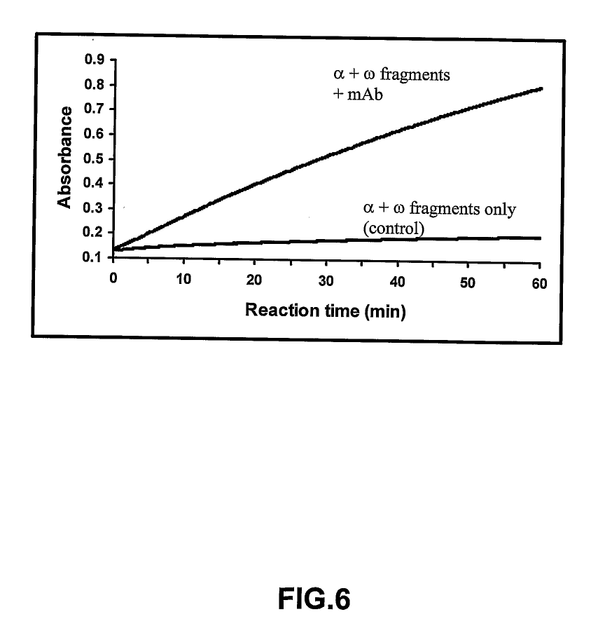

Results of an exemplary embodiment employing equimolar amounts of α and ω fragments of the invention are provided in FIG. 6.

It is shown in FIG. 6 that homogeneous in vino FEC can detect large analytes such as a monoclonal antibody (150 kDa) successfully at concentration of 33 nM. It was further determined to find the K_{D} of Ab concentration to be 18.43 nM.

### Example 3

### β-lactamase TEM1 Inhibitor Resistant Forced Enzyme Complementation (FEC) Homogeneous Assay

The procedures outlined herein describe the synthesis and characterisation of TEM1 fragments generated by splitting the full-length parental enzyme at amino acids 196/197 followed by introduction of a flexible linker (G₄S) and histidine tag (6×H) at the break-point termini. The subsequent α fragment (PB11) and ω fragment (PB13) were used to introduce point mutations in order to increase resistance to β-lactamase inhibitors, resulting in the generation of αM69LM182T fragment (PB11.12) and ωN276D fragment (PB13.3). DNA sequence data confirmed the presence of the flexible linker (G₄S) histidine tag (6×H) and point mutations. These fragment pairs were used to demonstrate forced enzyme complementation with an analyte (Ni²⁺) in the presence of β-lactamase inhibitors.

### Site-Directed Mutagenesis

Site-directed PCR mutagenesis of α (PB11) and ω (PB13) fragments were performed to generate the PB11.1 (αM69L), PB11.12 (a M69LM182T), PB11.13 (αM691M182T) and PB13.3 (ωN276D) constructs.

Briefly, α and ω fragment plasmid DNA was isolated and quantitated as described in Example 1 and used as the template in PCR amplified mutagenesis reactions as follows: 25 ng of template DNA was added to 125 ng forward primer (FEC55, FEC143, FEC145 or FEC147), 125 ng reverse primer (FEC56, FEC144, FEC146 or FEC148), 1× Reaction Buffer, 1 μl dNTP mix, 3 μl QuickSolution and ultra pure water to a final volume of 50 μl. PfuUltra HF DNA polymerase (2.5 U/μl) was then added and temperature cycling was performed with a Mastercycler ep gradient thermal cycler (Eppendorf). For introduction of the M69L, M69I and N276D point mutations PCR cycling was done as follows: denaturation at 95° C.×1 min, and 18 cycles of 95° C.×50 sec (denaturation), 60° C.×50 sec (annealing) and 68° C.×6 min (extension) with a final extension step of 68° C.×6 min at cycling completion. The PCR cycling conditions for introduction of the M182T mutation varied slightly with annealing done at 65° C.×50 sec using αM69L and αM69I fragments as templates and FEC55 and FEC56 forward and reverse primers respectively. PCR amplified mutagenesis reactions were subsequently digested with 1 μl DpnI restriction enzyme (10 U/μl) added directly to each reaction and incubated at 37° C. for 1 hour to digest parental non-mutated DNA. Two μl of Dpn I treated DNA from each sample reaction was then used to transform XL10-Gold Ultracompetent Cells as outlined in the QuickChange II XL-Site-Directed Mutagenesis Kit manufacturer's instruction manual (Rev# 124001e). E. coli transformants were screened as outlined previously, with resultant plasmid DNA sequenced to confirm correct insertion of point mutations. Plasmid DNA incorporating desired mutations was then used to transform BL21-Gold (DE3) competent cells as described for protein expression and purification.

### Purification and Characterisation of α and ω Fragments

All six enzyme fragments [α (PB11), αM69L (PB11.11), αM69LM182T (PB11.12), αM691M182T (PB11.13), ω(PB13), and ωN276D (PB13.3)] were expressed, purified and characterised in the same manner, in order to directly compare their kinetic properties in the absence and presence of β-lactamase inhibitors and to determine their signal to noise ratio upon forced enzyme complementation with Ni²⁺ (analyte) as described below.

### Expression of α and ω Fragments

Expression of fragments in BL21-Gold (DE3) was performed as described in Example 1.

### Extraction of Recombinant Proteins Under Denaturing Conditions

The pellet from a 250 ml overnight induction was lysed in 10 m/g (wet pellet weight) of lysis buffer (6 M GuHCl, 100 mM NaH₂PO₄, 10 mM Tris, 1 mM DTT pH 8 for the α fragment or 6 M GuHCl, 100 mM NaH₂PO₄, 10 mM Tris, pH 8 for the ω fragment), followed by a 1-hour incubation at 4° C. with shaking at 100 rpm. To enhance lysis, the suspension was sonicated in an ice bath for 5 cycles of 30 seconds on/30 seconds off using a Branson 250 sonifier. After sonication, the lysate was centrifuged at 12,000×g for 30 min (4° C.) and then passed through a 0.2 μm filter to remove cellular debris.

Immobilised Metal Affinity Chromatography (IMAC) and on-Column Refolding of α-Fragments

Recombinant His-tagged proteins were purified using a 1 ml HisTrap™ HP column (Amersham cat. 17-5247-01) under the control of an AKTA-FPLC (GE Healthcare) using the Unicorn 5.1 controller software (GE Healthcare) at 4° C. The HisTrap column was equilibrated with 10 column volumes (CV) of gradient buffer (8 M Urea, 100 mM NaH₂PO₄, 150 mM NaCl, 10 mM Tris, 200 mM L-arginine, 1 mM GSSG, 0.1 mM GSH, pH 8) at a flow rate of 1 ml/min. Cleared E. coli lysates were directly loaded onto the column using a 50 ml superloop to inject sample directly via the injection valve (INV-907) at 1 ml/min. Bound protein was refolded over a 50 column volume (CV) gradient from 8 M urea, 100 mM NaH₂PO₄, 150 mM NaCl, 10 mM Tris, 200 mM L-arginine, 1 mM GSSG, 0.1 mM GSH, pH 8 to 100 mM NaH₂PO₄, 150 mM NaCl, 10 mM Tris, 200 mM L-arginine, pH 8 at 1 ml/min. Contaminating protein was washed off the column with 10 CV of 20 mM imidazole, 50 mM NaH₂PO₄, 300 mM NaCl, pH 8. Histidine-tagged proteins were eluted with 10 CV of 500 mM imidazole, 50 mM NaH₂PO₄, 300 mM NaCl, pH 8 and analysed by PAGE, western blotting, gel filtration and mass spectroscopy.

Immobilised Metal Affinity Chromatography (IMAC) and on-Column Refolding of Co-Fragments

Recombinant His-tagged proteins were purified using a 1 ml HisTrap™ HP column (Amersham cat. 17-5247-01) under the control of an AKTA-FPLC (GE Healthcare) using the Unicorn 5.1 controller software (GE Healthcare) at 4° C. The HisTrap column was equilibrated with 10 CV of gradient buffer (8 M urea, 100 mM NaH₂PO₄, 10 mM Tris, 200 mM L-arginine, pH 7.5) at a flow rate of 1 ml/min. Cleared E. coli lysates were directly loaded onto the column using a 50 ml superloop to inject sample directly via the injection valve (INV-907) at 1 ml/min. Bound protein was refolded over a 50 CV gradient from 8 M urea, 100 mM NaH₂PO₄, 10 mM Tris, 200 mM L-arginine, pH 7.5 to 100 mM NaH₂PO₄, 10 mM Tris, 200 mM L-arginine, pH 7.5 at 1 ml/min. Contaminating protein was washed off the column with 10 CV of 20 mM imidazole, 50 mM NaH₂PO₄, 300 mM NaCl, pH 8. Histidine-tagged proteins were eluted with 10 CV of 500 mM imidazole, 50 mM NaH₂PO₄, 300 mM NaCl, pH 8 and analysed by PAGE, western blotting, gel filtration and mass spectroscopy.

### Enzyme Fragment Complementation

Purified α and ω fragments containing the 6-histidine-tag that can bind Ni²⁺, were used for kinetic studies to calculate the signal (Ni²⁺ forced enzyme complementation) to noise (spontaneous enzyme complementation without Ni²⁺) ratio. Different substrates, buffer additives and inhibitors were used to investigate and evaluate the signal to noise ratio of forced enzyme complementation.

### Nitrocefin Activity Assays and Panel Serum Screening

Nitrocefin (10 μL of a 4 mM stock solution prepared in 100% DMSO) was added to 190 μL reaction mixtures to give final concentrations of 0.75 M urea, 150 mM NaCl, 50 mM NaH₂PO₄ pH 7, 10-20 nM of each enzyme fragment (α and ω) and, where appropriate, 200 μM Ni²⁺. Assay components were mixed well by pipetting and incubated for 5 minutes at room temperature prior to substrate addition. The rate of nitrocefin hydrolysis was measured at 492 nm using a SpectraMax190 (Molecular Devices, USA) over a 30 min time frame. For serum assays, a panel of 50 individual sera were screened as above with the inclusion of serum at a final dilution of 1/200 prior in place of Ni²⁺. Final concentrations of serum assays were as follows: 0.6 M urea, 50 mM NaH₂PO₄, 150 mM NaCl, pH 7 with 10 nM of each α and ω fragments. Where appropriate, serum assays were spiked with 1.1 μM tazobactam and 2.8 μM tazobactam [2× and 5× the expected Cₘₐₓ serum concentration following an intravenous tazobactam dose (Wise, R., M. Logan, et al., 1991, Antimicrob Agents Chemother 35(6): 1081-4).

### Enzyme Kinetics of Inhibitor Resistant Mutants

Activity assays using Ni²⁺ were performed as above with the following exceptions: Nitrocefin (100 μL of various serial dilutions to produce final substrate concentrations ranging from 0 mM-1.6 mM) was added to 100 μL of the reaction mixture (in the absence of urea). Nitrocefin hydrolysis was measured by monitoring the reaction at 492 nm using a SpectraMax190 over 10 min at room temperature. The initial rate of reaction (first 10 readings, mOD/min) was plotted against the substrate concentration to determine the Kₘ and k_{cat} for each α and ω fragment pair. To determine the inhibitor IC₅₀ values in a Ni²⁺ assay, nitrocefin (10 μL of 2 mM nitrocefin in 100% DMSO) was added to reaction mixtures containing serial dilutions of each inhibitor to give final concentrations of 50 mM NaH₂PO₄, 150 mM NaCl, pH 7 10 μM tazobactam (Sigma Cat#T2820), 0-100 μM sulbactam (Molekula Prod#19590299) or 0-100 μM clavulanic acid (Molekula Prod#87644048), 25 nM of each α and ω fragment, 100 μM nitrocefin and 200 μM Ni²⁺ in a final volume of 200 μL. The rate of nitrocefin hydrolysis was measured at 492 nm using a SpectraMax190 for 10 min at room temperature. The initial rate of reaction (first 10 readings, mOD/min) was then plotted against each inhibitor concentration to determine the inhibitor IC₅₀ for each α and ω fragment pair.

### Results

TABLE 3
Inhibitor resistant construct kinetic analysis

| Reconstituted | | Vmax | Nitrocefin | Kcat |
|---|---|---|---|---|
| Enzyme | Fragment Pair | (mOD/min) | Km (M) | (s⁻¹) |
| TEM-1 (wt) | PB11/PB13 | 28.02 ± 0.41 | 44.82 + 2.193 | 5.1 |
| TEM-1N276D | PB11/PB13.3 | 30.86 ± 2.48 | 36.15 + 11.82 | 5.6 |
| TEM-33 | PB11.11/PB13 | 13.85 ± 0.87 | 259.1 + 38.83 | 2.5 |
| TEM-35 | PB11.11/PB13.3 | 8.524 ± 0.76 | 58.13 + 18.71 | 1.5 |
| TEM-33M182T | PB11.12/PB13 | 24.29 ± 2.07 | 106.4 + 27.96 | 4.4 |
| TEM33M182TN276D | PB11.12/PB13.3 | 25.57 ± 1.85 | 83.63 + 19.87 | 4.6 |

TABLE 4
β-lactamase inhibitor IC₅₀ determination

| Reconstituted | | Vmax | Tazobactam |
|---|---|---|---|
| Enzyme | Fragment Pair | (mOD/min) | IC₅₀ (nM) |
| TEM-1 | PB11/PB13 | 83.51 ± 2.87 | 102.3 ± 11.13 |
| TEM-1N276D | PB11/PB13.3 | 95.77 ± 4.75 | 175.9 ± 24.21 |
| TEM-33 | PB11.11/PB13 | 44.04 ± 1.51 | 1200 ± 15.63 |
| TEM-35 | PB11.11/PB13.3 | 33.31 ± 0.81 | 828.6 ± 72.69 |
| TEM-33M182T | PB11.12/PB13 | 123.0 ± 2.81 | 1529 ± 144.3 |
| TEM-33M182TN276D | PB11.12/PB13.3 | 83.09 ± 2.76 | 1208 ± 168.9 |
| | | | Clavulanic |
| Reconstituted | | Vmax | Acid |
| Enzyme | Fragment Pair | (mOD/min) | IC₅₀ (μM) |
| TEM-1 | PB11/PB13 | 60.78 ± 3.42 | 1.682 ± 0.29 |
| TEM-1N276D | PB11/PB13.3 | 91.25 ± 3.85 | 2.088 ± 0.27 |
| TEM-33 | PB11.11/PB13 | 28.78 ± 1.78 | 30.91 ± 8.51 |
| TEM-35 | PB11.11/PB13.3 | 24.47 ± 0.70 | 246.6 ± 69.03 |
| TEM-33M182T | PB11.12/PB13 | 86.33 ± 4.51 | 46.71 ± 12.28 |
| TEM-33M182TN276D | PB11.12/PB13.3 | 89.76 ± 2.76 | 59.15 ± 9.58 |
| Reconstituted | | Vmax | Sulbactam |
| Enzyme | Fragment Pair | (mOD/min) | IC₅₀ (μM) |
| TEM-1 | PB11/PB13 | 59.57 ± 2.77 | 2.675 ± 0.38 |
| TEM-1N276D | PB11/PB13.3 | 165.2 ± 8.64 | 2.944 ± 0.75 |
| TEM-33 | PB11.11/PB13 | 28.12 ± 1.11 | 24.59 ± 4.16 |
| TEM-35 | PB11.11/PB13.3 | 38.95 ± 0.69 | 47.05 ± 6.73 |
| TEM-33M182T | PB11.12/PB13 | 89.83 ± 2.93 | 32.51 ± 4.80 |
| TEM-33M182TN276D | PB11.12/PB13.3 | 141.4 ± 1.95 | 52.00 ± 6.07 |

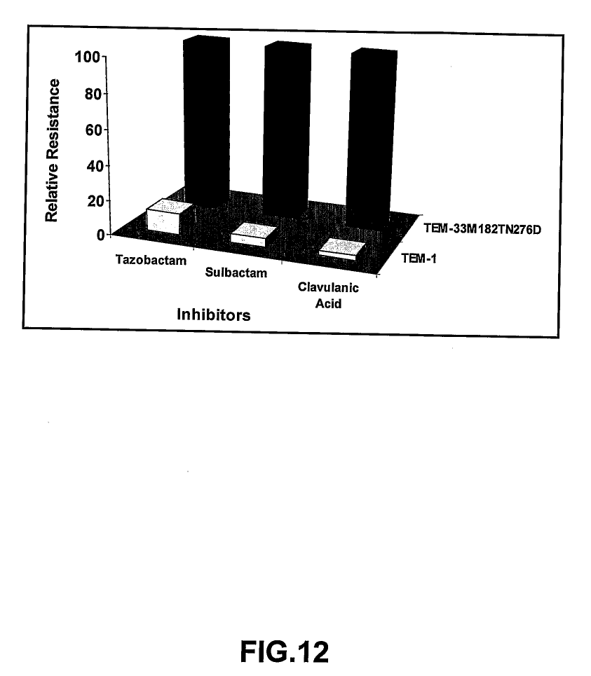

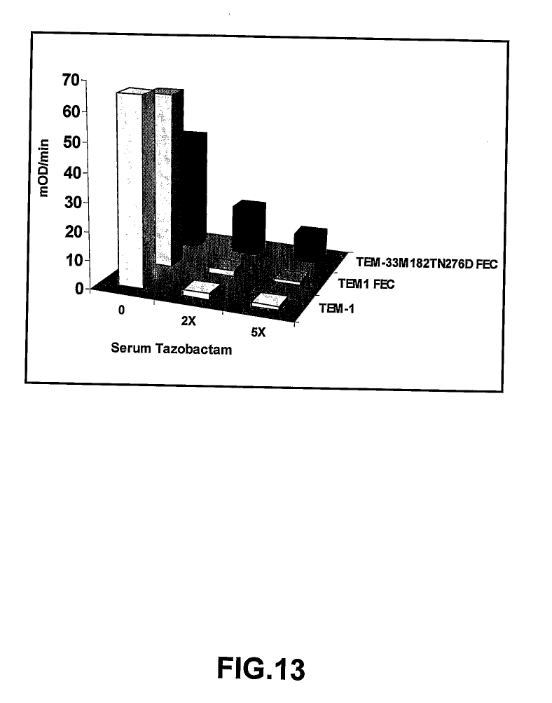

(see also FIGS. 12 and 13)

These results demonstrate that beta-lactamase fragments incorporating the above mutations have increased resistantance to the inhibitors. These findings will be applied to the subsequent beta-lactamase fragments to be used in homogeneous in vitro FEC assays for detection of HSV-1 and HSV-2 IgG in serum samples (Example 4).

### Example 4

### β-Lactamase TEM1 Inhibitor Resistant Forced Enzyme Complementation (FEC) Assay for the Detection of Disease Specific IgG in Patient Sera

Examples 1, 2 and 3 describe the synthesis and characterisation of TEM1 fragments generated by splitting the full-length parental enzyme (PB3) at amino acids 196/197 followed by introduction of a flexible linker (G₄S) and histidine tag (6×H) at the break-point termini. The subsequent α-fragment (PB11) and ω-fragment (PB13) were used to introduce point mutations in order to increase resistance to β-lactamase inhibitors, resulting in the generation of αM69L fragment (PB11.11), αM69LM182T fragment (PB11.12), αM691M182T fragment (PB11.13) and ωN276D fragment (PB13.3). DNA sequence data confirmed the presence of the flexible linker (G₄S) histidine tag (6×H) and point mutations. These fragment pairs were used to demonstrate forced enzyme complementation (FEC) with an analyte (Ni²⁺, Zn²⁺ or 6×H monoclonal antibody binding to the histidine tag) in the presence of β-lactamase inhibitors (potentially present in the sera of patients being administered antibiotics).

In this example, the enzyme fragments were fused to analyte binding moieties (HSV-1 truncated antigen, HSV-2 truncated antigen and protein-G subunit) for the detection of disease specific IgG antibodies in patient sera.

### Methods

### DNA Constructs

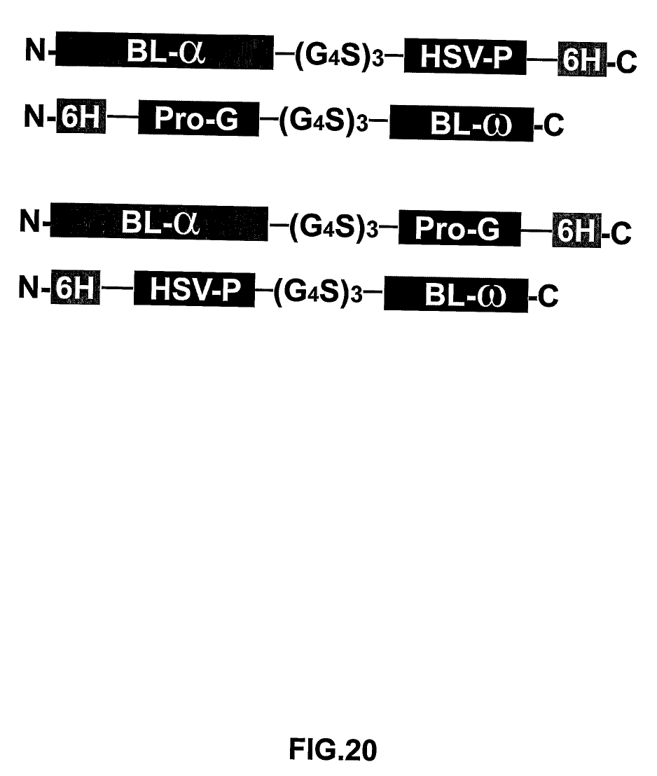

Schematic representations of the β-lactamase constructs used in this study are illustrated in FIG. 20. The corresponding oligonucleotides (Sigma-Genosys) used for the construction of all plasmids are listed in Table 5. The first construct, expressing C-terminal hexa-histidine-tagged (CHis) full-length β-lactamase (not including the N-terminal secretory sequence), was created by PCR amplification of the pUC18 Bla gene using PAN1 and PAN2 primers. The PCR product was digested with NdeI and XhoI and ligated into pET-26b(+) (Merck) to give pET-BL. Using this construct as a template, PAN1/PAN3 and PAN4/PAN5 were used to amplify two adjacent regions of the bla gene for expression of BLα (residues 25-196) bearing a CHis tag fused via a G₄S linker; and BLω (residues 197-290) with an N-terminal hexahistidine tag, also connected via a G₄S linker. Each PCR product was digested with NdeI and XhoI and ligated into pET-26b(+) to give pET-BLα and pET-BLω. Sequences expressing longer (G4S)₃ linkers were incorporated into both pET-BLα and pET-Blω, giving pET-BLα(G₄S)₃ and pET-BLω(G₄S)₃, to serve as precursors for future constructs. These were introduced by site-directed mutagenesis (QuickChange II XL-Site-Directed Mutagenesis Kit) using the primers PAN6/PAN7 and PAN8/PAN9, respectively.

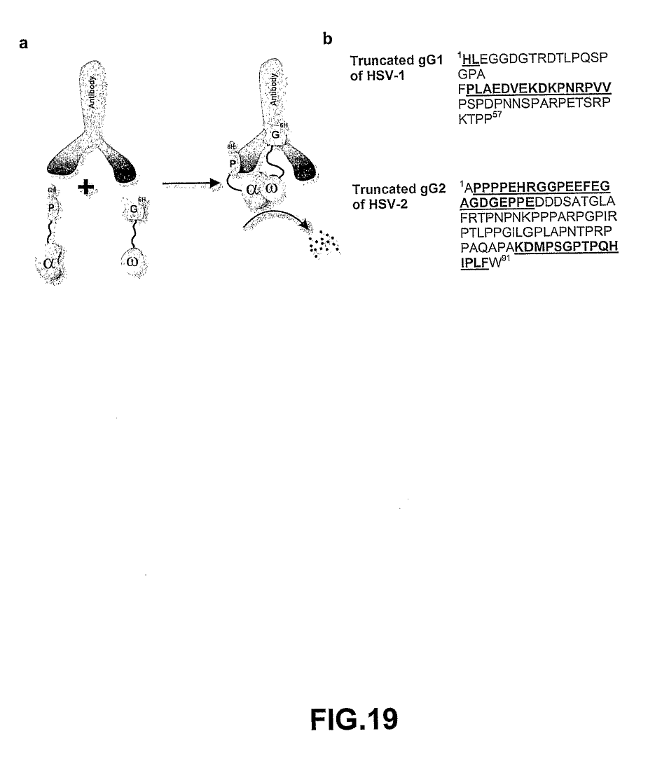

Subsequent fragments of β-lactamase were engineered to harbour three different types of analyte-binding moieties: peptides comprising either epitopes of glycoprotein G1 (gG1; HSV-1 antigen; FIG. 19b) or glycoprotein G2 (gG2; HSV-2 antigen; FIG. 19b and the C2 domain of protein G (ProG). The first two DNA constructs that were generated, express BLα fused to either an HSV-1 (pET-BLα-HSV1) or an HSV-2 specific antigenic peptide (pET-BLα-HSV2). The construction of these fragments is described as follows. First, overlap extension PCR using oligonucleotides PAN10, PAN11, PAN12, and PAN13 (Table 5), was performed in order to generate a megaprimer encoding the HSV-1 antigen. The second PCR utilized pET-BLω(G₄S)₃ as a template, the megaprimer from the first PCR, and PAN14 to give the BLα-HSV1 gene. This second PCR product was digested with NdeI and XhoI and ligated into pET-26b(+). The construct pET-BLα-HSV2, encoding BLα-HSV2 was made in the same way using oligonucleotides PAN10, PAN15, PAN16, PAN17, PAN18 and PAN19 for overlap extension PCR.

To simplify the engineering of subsequent fusion constructs, universal BLα and BLω fusion genes were synthesized by DNA2.0 (Menlo Park, U.S.A) having restriction endonuclease sites incorporated between the enzyme fragment, linker, and binding moieties to enable the substitution of various domain sequences. The universal BLω construct, pET-BLω-ProG, was designed to have BamHI, SpeI and NheI sites inserted between the ProG, (G₄S)₃ linker and BLω encoding domains with NdeI and XhoI sites on either end for ligation into the NdeI/XhoI site of pET-26b(+). The sequence encoding the C2 IgG-binding domain of Streptococcus strain G148 Protein G was obtained by back-translation of the published amino acid sequence (Galich et al., 2002, Protein Eng. 15(10): 835-42) with an E. coli codon usage table, using Vector NTI (Invitrogen). The universal BLα construct, pET-BLα-ProG, was created using a similar approach. The gene sequence was designed to have KpnI, BamHI and SpeI sites between the BLα sequence, (G₄S)₃ linker and antigen encoding moieties with NdeI and XhoI sites on either end for ligation into the NdeI/XhoI site of pET-26b(+). For this construct, the sequence encoding ProG was excised from pET-BLω-ProG and ligated into the BamHI/SpeI site of the universal BLα construct.

As an alternative to BLα-HSV1 and BLα-HSV2, we used the universal BLω construct to create complementary BLω-HSV1 and BLα-HSV2 constructs by substituting the ProG sequence with the respective HSV-1 and HSV-2 antigenic peptide sequences. The sequence encoding the HSV-1 peptide was PCR-amplified from pET-BLω-HSV1 using oligonucleotides PAN20 and PAN21 to incorporate flanking BglII and SpeI sites. Similarly, the HSV-2 peptide sequence was PCR-amplified from pET-BLα-HSV2 using oligonucleotides PAN22 and PAN23 to incorporate flanking BamHI and SpeI sites. PCR products were then ligated into the BamHI/SpeI site of pET-BLω-ProG, to give pET-BLω-HSV1 and pET-BLω-HSV2.

The point mutation N276D was introduced into the ω fragment sequence of pET-BLω-ProG, pET-BLω-HSV1, and pET-BLω-HSV2 by site-directed mutagenesis (QuickChange II XL-Site-Directed Mutagenesis Kit) using oligonucleotides PAN24 and PAN25. All cloned and mutated inserts were sequenced by the Australian Genome Research Facility (AGRF, Brisbane, Australia).

TABLE 5
Primer sequences

| Oligonucleotide | Sequence (5′ to 3′) | SEQ ID NO: |
|---|---|---|
| PAN1 | CTTTTCCCTGTCCATATGCACCCAGAAACGCTGGTGAAAG | 41 |
| PAN2 | GGAATACCGGTTCTCGAGCCAATGCTTAATCAGTGAGGCACC | 42 |
| PAN3 | AACCGGTATTCCCTCGAGTCAGTGATGATGATGGTGATGCGAGCCACC | 43 |
| | TCCACCGCCAGTTAATAGTTTGCGCAACGTTGTTGC | |
| PAN4 | CTTTTCCCTGTCCATATGCATCACCATCATCATCACGGTGGAGGTGGCT | 44 |
| | CGGAACTACTTACTCTAGCTTCCCGGCAAC | |
| PAN5 | AACCGGTATTCCCTCGAGTCACCAATGCTTAATCAGTGAGGCACC | 45 |
| PAN6 | GGAAGCGGCGGAGGTGGTTCTGGTGGTGGAGGCTCGCATCACCATCA | 46 |
| | TCATCACTGAC | |
| PAN7 | TCCACCACCAGAACCACCTCCGCCGCTTCCACCTCCACCGCCAGTTAA | 47 |
| | TAG | |
| PAN8 | GGTGGTGGAGGCAGCGGCGGAGGTGGTTCTGGTGGAGGTGGCTCGG | 48 |
| | AACTAC | |
| PAN9 | AGAACCACCTCCGCCGCTGCCTCCACCACCGTGATGATGATGGTGATG | 49 |
| | CATATG | |
| PAN10 | CCTCTCGAGTCAGTGATGATGATGATGGTGACTACCGCCACCGCC | 50 |
| PAN11 | CGGAGGTGGTTCTGGTGGTGGAGGCTCGCATCTTGAAGGCGGCGATG | 51 |
| | GCACAAGAGATACCCTTCCTCAGAGTCCAGGTCCGGCG | |
| PAN12 | GGATCTGGGCTCGGCACTACTGGTCTGTTCGGTTTATCTTTCTCCACAT | 52 |
| | CTTCCGCCAGTGGAAACGCCGGACCTGGACTCTGAG | |
| PAN13 | GTAGTGCCGAGCCCAGATCCGAACAACAGCCCAGCGAGACCGGAAACC | 53 |
| | AGCAGACCGAAAACCCCTCCCGGCGGTGGCGGTAGTC | |
| PAN14 | CGCCATATGCACCCAGAAACGCTGGTGAAAGTAAAAGATGCTGAAG | 54 |
| PAN15 | CTGGTGGTGGAGGCTCGGCTCCTCCTCCTCCTGAACATCGTGGCGGC | 55 |
| | CCGGAAGAATTTGAAGGCGCTGGCGATGGC | |
| PAN16 | CGGGTTCGGGGTACGAAACGCCAGACCAGTTGCACTATCATCATCTTC | 56 |
| | CGGCGGTTCGCCATCGCCAGCGCCTTC | |
| PAN17 | GTTTCGTACCCCGAACCCGAACAAACCGCCGCCTGCACGTCCTGGTCC | 57 |
| | TATTCGTCCGACCCTGCCGCCTGGCATTC | |
| PAN18 | CTTTCGCCGGAGCTTGTGCAGGAGGACGAGGGGTGTTCGGAGCCAGC | 58 |
| | GGACCTAGAATGCCAGGCGGCAGGGTC | |
| PAN19 | GCACAAGCTCCGGCGAAAGATATGCCGAGTGGCCCGACCCCGCAGCA | 59 |
| | TATTCCTCTGTTTTGGGGCGGTGGCGGTAGTCAC | |
| PAN20 | GTTTAACTTCGCAGATCTCATCTGGAAGGCGGCGATGGCAC | 60 |
| PAN21 | CCACTAGTCGGCGGGGTTTTCGGGCGGCTGGTTTCC | 61 |
| PAN22 | CGCGGATCCGCGCCGCCGCCGCCGGAACATC | 62 |
| PAN23 | CCACTAGTCCAAAACAGCGGAATATGCTGCGGGGTCGGGCCGCTC | 63 |
| PAN24 | ATGGATGAACGAGATAGACAGATCGCTGAG | 64 |
| PAN25 | CTCAGCGATCTGTCTATCTCGTTCATCCAT | 65 |

### HSV-1 and HSV-2 Antigenic Peptide Design

In order to detect and discriminate between antibodies against HSV-1 and HSV-2 in serum, two type-specific HSV antigenic peptides were designed as analyte binding moieties. The HSV-1 specific peptide (FIG. 19b) is comprised of residues 92-148 of glycoprotein G1 (gG1). This region of gG1 contains an immunodominant epitope (residues 112-127, and two key amino acids within a second epitope known to confer an HSV type-1 specific response in humans. The HSV-2 specific peptide (FIG. 19b) is composed of residues 551-641 of glycoprotein G2 (gG2) and is comprised of two immunodominant epitopes (residues 561-578 and 626-640) known to confer an HSV type-2 specific response in humans.

### Expression and Purification of α and ω Reporter Fragments

All enzyme fragments [α fragment (PB11), αM69L fragment (PB11.11), αM69LM182T fragment (PB11.12), αM691M182T fragment (PB11.13), α-HSV-1 fragment, α-HSV-2 fragment, α-Protein-G fragment and w fragment (PB13), ωN276D fragment (PB13.3), HSV-1-ω fragment and HSV-2-ω fragment] were expressed (as described in Example 1) and purified under denaturing conditions. Protein-G-ω fragment was purified under native conditions. All purified proteins were characterised and assayed in a similar manner.

### Extraction of Recombinant Proteins Under Denaturing Conditions

The pellet from a 250 ml overnight induction was lysed in 10 ml/g (wet pellet weight) of lysis buffer (6M GuHCl, 100 mM NaH₂PO₄, 10 mM Tris, 1 mM DTT pH 8) for α fragment (PB11, PB11.11, PB11.12 and PB11.13) and 6 M GuHCl, 100 mM NaH₂PO₄, 10 mM Tris, pH 8, for ω fragment (PB13 and PB13.3). The α-fragment-analyte binding moiety fusions (BLαHSV-1, BLαHSV-2 and BLαProG) were lysed in 6 M GuHCl, 100 mM NaH₂PO₄, 200 mM L-arginine, 20 mM Imidazole, 2 mM DTT pH 8. The analyte binding moiety-ω-fragment fusions (BLωHSV1 and BLωHSV2) were lysed in 6 M GuHCl, 100 mM NaH₂PO₄, 200 mM L-arginine, 20 mM Imidazole, pH 8. Following a 1-hour incubation at 4° C. with shaking at 100 rpm, each suspension was sonicated in an ice bath for 5 cycles of 30 seconds on/30 seconds off using a Branson 250 sonifier. After sonication, lysates were centrifuged at 12,000×g for 30 min (4° C.) and then passed through a 0.2 μm filter.

### Extraction and Purification of BLωProG Under Native Conditions

The pellet from a 250 ml overnight induction was resuspended in native lysis buffer (10 mM imidazole, 50 mM NaH₂PO₄, 300 mM NaCl pH8) at 5 ml per gram wet weight. Lysozyme (Sigma) was added to 1 mg/ml and the suspension was incubated on ice for 30 min. The lysate was sonicated in an ice bath for 5 cycles of 30 seconds on/30 seconds off using a Branson 250 sonifier (output 6 and 70% duty). Lysate was centrifuged at 10,000×g for 30 min at 4° C. Supernatant was decanted and passed through a 0.2 μm filter. A protease inhibitor cocktail (Pierce) was added to the filtrate as directed by the manufacturer. BLωProG was purified under native conditions using Ni-NTA resin (Qiagen cat# 30210). One ml of Ni-NTA was added to lysate and mixed gently by shaking (100 rpm) at 4° C. for 1 hour. Lysate-Ni-NTA mixture was poured into a 1×10 cm column and washed with 16 ml of native wash buffer (20 mM imidazole, 50 mM NaH₂PO₄, 300 mM NaCl pH8). Bound protein was eluted with 10 ml of native elution buffer (250 mM imidazole, 50 mM NaH₂PO₄, 300 mM NaCl pH8) in 1 ml fractions.

### Immobilised Metal Affinity Chromatography (IMAC) and On-Column Refolding of PB11, PB11.11, PB11.12 and PB11.13

Recombinant 6×H-tagged proteins were purified using a 1 ml HisTrap™ HP column (Amersham cat. 17-5247-01) under the control of an AKTA-FPLC (GE Healthcare) using the Unicorn 5.1 controller software (GE Healthcare) at 4° C. The HisTrap column was equilibrated with 10 column volumes (CV) of gradient buffer (8 M urea, 100 mM NaH₂PO₄, 150 mM NaCl, 10 mM Tris, 200 mM L-arginine, 1 mM GSSG, 0.1 mM GSH, pH 8) at a flow rate of 1 ml/min. Cleared E. coli lysates were directly loaded onto the column using a 50 ml superloop to inject sample directly via the injection valve (INV-907) at 1 ml/min. Bound protein was refolded over a 50 CV gradient from 8 M Urea, 100 mM NaH₂PO₄, 150 mM NaCl, 10 mM Tris, 200 mM L-arginine, 1 mM GSSG, 0.1 mM GSH, pH 8 to 100 mM NaH₂PO₄, 150 mM NaCl, 10 mM Tris, 200 mM L-arginine, pH 8 at 1 ml/min. Contaminating protein was washed off the column with 10 CV of 20 mM imidazole, 50 mM NaH₂PO₄, 300 mM NaCl, pH 8. Histidine-tagged proteins were eluted with 10 CV of 500 mM imidazole, 50 mM NaH₂PO₄, 300 mM NaCl, pH 8 and collected into 1 ml fractions using a FRAC950 fraction collector (GE Healthcare). Proteins were further analysed by PAGE, western blotting, gel filtration and mass spectroscopy.

### Immobilised Metal Affinity Chromatography (IMAC) and On-Column Refolding of PB13 and PB13.3

Recombinant 6×H-tagged proteins were purified using a 1 ml HisTrap™ HP column (Amersham cat. 17-5247-01) under the control of an AKTA-FPLC (GE Healthcare) using the Unicorn 5.1 controller software (GE Healthcare) at 4° C. The HisTrap™ column was equilibrated with 10 CV of gradient buffer (8 M Urea, 100 mM NaH₂PO₄, 10 mM Tris, 200 mM L-arginine, pH 7.5) at a flow rate of 1 ml/min. Cleared E. coli lysates were directly loaded onto the column using a 50 ml superloop to inject sample directly via the injection valve (INV-907) at 1 ml/min. Bound protein was refolded over a 50 CV gradient from 8 M urea, 100 mM NaH₂PO₄, 10 mM Tris, 200 mM L-arginine, pH 7.5 to 100 mM NaH₂PO₄, 10 mM Tris, 200 mM L-arginine, pH 7.5 at 1 ml/min. Contaminating protein was washed off the column with 10 CV of 20 mM imidazole, 50 mM NaH₂PO₄, 300 mM NaCl, pH 8. Histidine-tagged proteins were eluted with 10 CV of 500 mM imidazole, 50 mM NaH₂PO₄, 300 mM NaCl, pH 8 and collected into 1 ml fractions.

Immobilised Metal Affinity Chromatography (IMAC) and on-Column Refolding of BLα-HSV1, BLα-HSV2, BLω-HSV1, BLω-HSV2 and BLαProG

Fusion proteins were purified using a 1 ml HisTrap™ HP column under the control of an AKTA-Purifier (GE Healthcare) using the Unicorn 5.1 controller software (GE Healthcare) at 4° C. The HisTrap column was equilibrated with 10 CV of gradient buffer (8 M Urea, 100 mM NaH₂PO₄, 200 mM L-arginine, pH 8) at a flow rate of 1 ml/min. Cleared E. coli lysates were directly loaded onto the column using a 50 ml superloop to inject sample directly via the injection valve (INV-907) at 1 ml/min. Bound protein was refolded over a 20 CV gradient from 8 M Urea, 100 mM NaH₂PO₄, 200 mM L-arginine, pH 8 to 100 mM NaH₂PO₄, 200 mM L-arginine, pH 8 at 1 ml/min. Contaminating protein was washed off the column with 10 CV of 50 mM imidazole, 100 mM NaH₂PO₄, 300 mM NaCl, pH 7.5, followed by a second 10 CV wash of 100 mM imidazole, 100 mM NaH₂PO₄, 300 mM NaCl, pH 7.5. Histidine-tagged proteins were eluted with 10 CV of 500 mM imidazole, 100 mM NaH₂PO₄, 300 mM NaCl, pH 7.5 and collected into 1 ml fractions. Fractions containing the protein peak (2.5 ml) were pooled and buffer exchanged into 50 mM NaH₂PO₄, 50% glycerol using a PD10 column (GE Healthcare) and stored at −20° C.

### Optimization of Linker Length for the BLω-ProG Fusion Construct

To make the BLω-ProG construct with a shorter (G₄S)₂ or longer (G₄S)₄ interdomain linker, the respective gene sequences were synthesized by DNA2.0 and ligated into the NdeI/XhoI site of pET-26b(+). Both fragments were purified under native conditions as per the original BLω-ProG. Assays were performed with the three BLω-ProG fragments in combination with BLα-HSV1, using either pooled serum from HSV1-positive individuals or hyper-immune sera from a rabbit immunized with the HSV-1 antigenic peptide as the source of analyte. For both assays, HSV-1/2 negative serum was included to indicate the level of background complementation. Serum was added to a final concentration of 1:100 in a 200 μl reaction that consisted of BLα-HSV 1 (5 nM), BLω-ProG (5 nM), 0.5 M Urea, 150 mM NaCl, 50 mM sodium phosphate buffer pH 7 and 100 μM nitrocefin. Reactions were incubated for 10 min at RT prior to measuring the kinetics of nitrocefin hydrolysis at 492 nm over 40 min at RT.

### β-Lactamase-Based FEC Assay for HSV-1 and HSV-2-Specific IgG.

Fifty serum samples from either normal individuals or those with proven HSV-1 or HSV-2 infections were tested. Four different combinations of fragments including (1) BLα-HSV1/BLω-ProG, (2) BLα-HSV2/BL-ProG, (3) BLω-HSV1/BLα-ProG, and (4) BLω-HSV2/BLα-ProG were tested with each of the 150 patient sera (50 HSV-2 positive/HSV-1 negative; 50 HSV-1 positive/HSV-2 negative; 50 HSV-1/HSV-2 negative). Sera (20 μL of 1:20 dilution in 50 mM sodium phosphate buffer pH 7), followed by nitrocefin (Merck) (20 μL of 1 mM nitrocefin in 5% DMSO, 50 mM sodium phosphate buffer pH 7) was added to 1604 of the homogeneous reaction mixture in a 96-well plate (Greiner) to give final concentrations of 1:200 patient sera, 100 μM nitrocefin, 0.5% DMSO, 5 nM BLα, 5 nM BLω, 0.5 M urea, 150 mM NaCl, 50 mM sodium phosphate buffer pH 7. Reaction mixtures were incubated for 15 minutes at RT on a platform rocker prior to measuring the rate of nitrocefin hydrolysis at 492 nm over 60 min at RT. Kinetics results were obtained and analyzed using SoftMaxPro software (Molecular Devices). The rates (mOD·min⁻¹) were calculated over the first 20 minutes of the assay.

### Results

### Optimization of Linker Length for BLω-ProG

We compared the efficacy of using either (G₄S)₂, (G₄S)₃ or (G₄S)₄ interdomain linkers within BLω-ProG. Complementation assays were conducted in which the three BLω-ProG variants and BLα-HSV1 were combined with either rabbit hyper-immune serum (anti-HSV-1 peptide) or HSV-1/2 negative serum as the model analyte and its control, respectively. Assays with the hyper-immune serum showed that BLω-ProG with a (G₄S)₃ linker gave the highest level of activity and therefore highest signal to background ratio. These assays demonstrate that BLω-ProG with (G₄S)₂ and (G₄S)₃ linkers give higher levels of activity compared to those with a larger (G₄S)₄ linker. All other BLω-ProG fragments used for FEC assays in this study incorporated a (G₄S)₃ linker.

### Specificity of BLω-ProG

To confirm that the Protein G moiety of BLω-ProG was functional and suitable for testing in our HA format we assessed its binding kinetics for human IgG using surface plasmon resonance. Analysis of the sensorgram data indicated that BLω-ProG has a dissociation constant (K_{D}) of 81 nM, which is equivalent to the published affinity of the C2 domain of protein G (93 nM), also determined using SPR. Although BLω-ProG has a slower rate of association than the C2 domain, this is compensated by a slower rate of dissociation. A commercially available recombinant protein G, truncated to eliminate binding to human serum albumin, was included as a positive control. This recombinant Protein G had a lower affinity of binding to human IgG (K_{D}=483 nM) than BLω-ProG when tested under the same conditions.

### Analyte Quantitation Using β-Lactamase-Based FEC

In order to determine the concentration-response of the in-vitro FEC format described here, BLα-HSV1 and BLω-ProG were assayed together in the presence of a model analyte. In this case, a mouse monoclonal anti-histidine Ab (anti-His MAb) was used as the model analyte since both enzyme fragments carry a hexahistidine tag on the proximal end of the β-lactamase split-point termini. The resultant curve (data not shown) displays a classical sigmoidal fit consistent with single-site saturable binding, confirming the ability of the assay format to distinguish between high and low analyte concentrations in a proportional manner. This implies that the assay can be used to quantitate analyte concentrations as long as the analyte concentration falls within the detection range. As expected, the control analyte (monoclonal anti-glutathione-S-transferase Ab; anti-GST MAb) did not produce a response curve, demonstrating the fidelity of the assay in the presence of antibody.

### Homogeneous Assay Using Beta-Lactamase-Based FEC for the Detection of HSV-1 and HSV-2

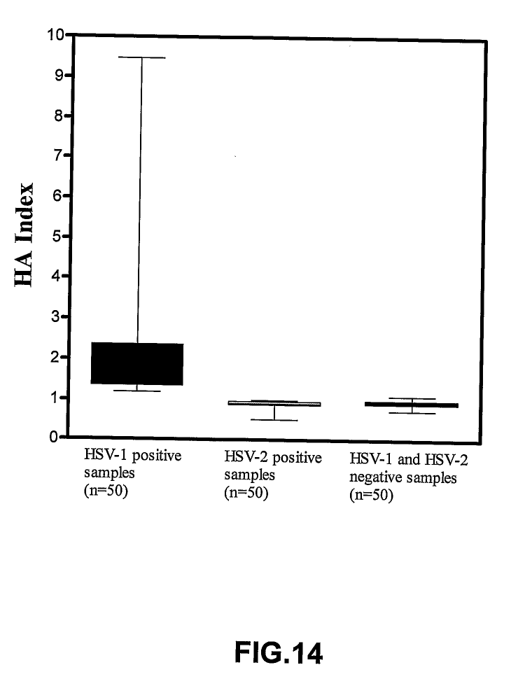

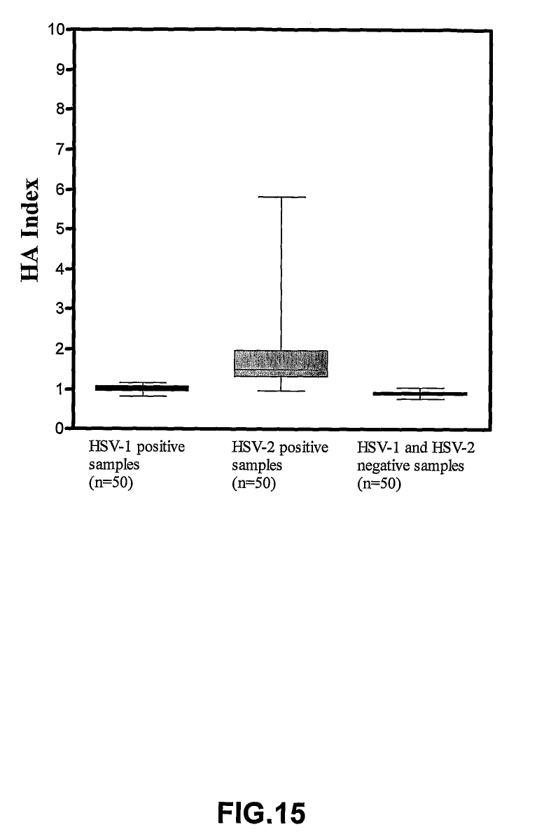

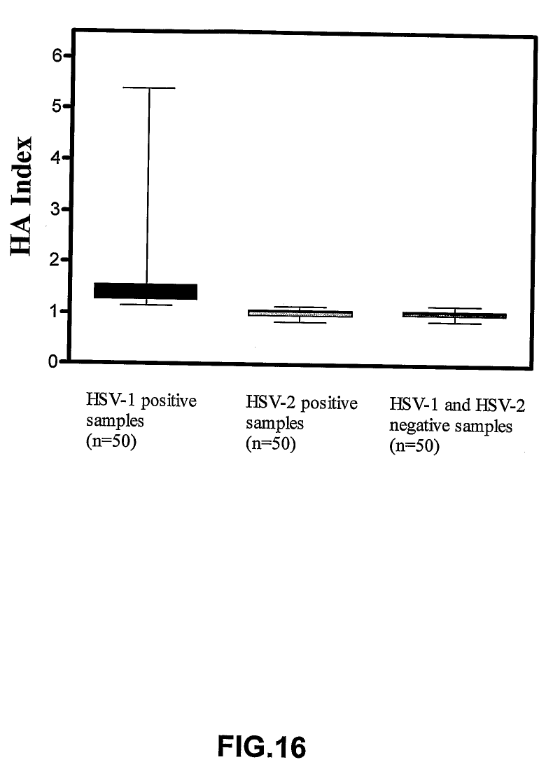

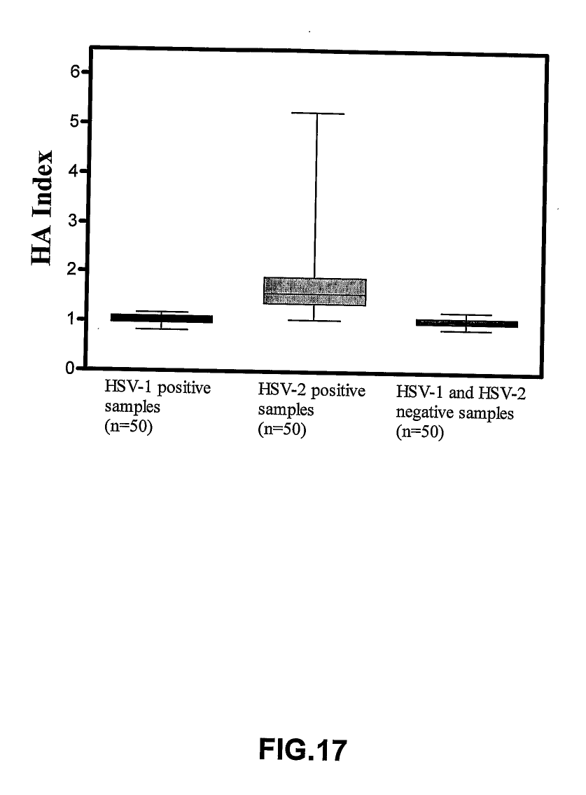

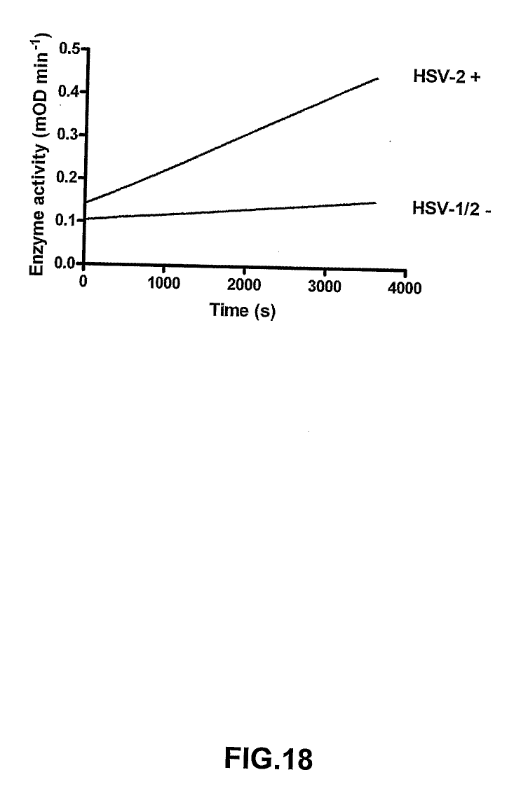

To test the performance of our homogeneous assays, we assayed 150 patient serum samples from Brisbane, Australia (50 HSV-1 positive/HSV-2 negative; 50 HSV-2 positive/HSV-1 negative; 50 HSV-1/HSV-2 negative) and compared our results with existing commercial assays, HerpeSelect1 and 2 ELISA IgG (Focus Diagnostics, USA) that were used as the internal standard. FIGS. 14 to 17 illustrate that beta-lactamase-based FEC can successfully detect type-specific HSV antibodies with high sensitivity and specificity. The results for each set of 50 assays (3 sets of assays for each of 4 different combinations of fragments-performed in triplicate over 3 days) were normalized against a sample of pooled serum (n=4) testing negative for HSV-1/2. FIG. 18 illustrates typical rates of nitrocefin hydrolysis by BLα-HSV2/BLω-ProG in serum from an HSV negative patient (0.85 mOD·min⁻¹) or from an individual with high HSV-2 positive serum (5.14 mOD·min⁻¹). Similar rates of hydrolysis were obtained with BLα-HSV1/BLω-ProG in HSV-1 high positive sera. The choice of fragment (BLα or BLω) for a given fusion partner (ProG, HSV-1, or HSV-2 specific peptides) had little effect on the overall results of the assay, although the activity of BLα-ProG/BLω-HSV1 (FIG. 14) is slightly higher overall than BLα-HSV1/BLω-ProG (FIG. 17). High serum:dilutions (1:200) in combination with low fragment concentrations (5 nM) in assays containing 150 mM NaCl and 0.5 M urea were used to maintain low background activity in control samples (HSV negative serum). Although the serum samples were assayed over a period of 75 minutes (including a 15 minute lag period following substrate addition), depending on the sample, color change could be observed within the first 25 minutes following substrate addition.

### Discussion

We have developed a new FEC-based homogeneous assay that can potentially be used for the detection of a broad variety of biomarkers. The system does not require dedicated instrumentation and, due to its simplicity, can be further developed for point-of-care use. To provide proof-of-concept, we developed an assay for the detection of type-specific HSV IgG using E. coli β-lactamase. The gene was split in two and engineered to express one fragment fused to either a HSV-1 or HSV-2-specific peptide antigen; the other to a single domain of protein G. All of the β-lactamase fragments were easily purified in high quantities and assayed in buffer simply by mixing two complementary fragments together in the presence of analyte and substrate. We have demonstrated that the β-lactamase-based FEC assay can detect large analytes in serum with limited cross-reactivity, interference, and inhibition, by identifying antibodies against HSV-1 and HSV-2 in human serum with high sensitivity and specificity.

Contradictory to previous reports of PCAs using either the same or different reporter enzymes, we have consistently observed background activity as a result of the spontaneous assembly of complementary fragments in the absence of analyte. This disagreement can be explained by the differences in the underlying nature of the two assays. PCAs are most commonly used to detect protein-protein interactions in vivo where the concentration of enzyme fragments can be as low as 25 molecules per cell (˜fM range). In contrast, our in vitro FEC-based assay uses fragment concentrations as high as 5 nM, thereby increasing the likelihood of spontaneous re-association. Nevertheless, the preliminary assays reported here generate a signal which is, on average, at least two-fold higher than the background and depending on the concentration of analyte in solution, we can achieve a signal to noise ratio as high as 15 in human serum. In the future, we aim to focus on producing fragments with much lower affinity for each other which will, in turn, lower both the limit of analyte detection and background noise whilst increasing the dynamic range of the assay.

There are few commercially available, reliable, type-specific serological tests for the diagnosis of HSV infection, and the most common of these are based on ELISA As part of our assay development strategy, we were successful in designing novel HSV type-specific antigens that, when fused to reporter enzyme fragments, are both sensitive and highly specific for the detection of antibodies against HSV-1 or HSV-2. The HSV-1 specific peptide contains an immunodominant region of gG1 in addition to two key amino acids that are known to elicit a HSV type-1 specific response in humans. Similarly, the HSV-2 specific peptide contains two immunodominant epitopes of gG2 that are known to elicit a HSV type-2 specific response in humans.

The FEC-based homogeneous EIA reported here is simple and could be easily automated for the detection of disease-specific biomarkers in real-time. Real-time analyte detection (sensor) provides greater dynamic range than end-point detection and is inherently quantitative for applications in the life sciences.

The various features and embodiments of the present invention, referred to in individual sections above apply, as appropriate, to other sections, mutatis mutandis. Consequently features specified in one section may be combined with features specified in other sections, as appropriate.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The references specified in the above text are incorporated herein by reference to the extent that they supplement, explain, provide a background for, or teach methodology, techniques, and/or compositions employed herein.

1-21. (canceled)

22. A method of assaying for the presence of an analyte, the method comprising the steps of:
(a) obtaining a sample to be tested for the presence of an analyte of interest;
(b) obtaining a purified first reporter fragment pair member comprising an interactor domain with affinity for the analyte;
(c) obtaining a purified second reporter fragment pair member comprising an interactor domain with affinity for the analyte of interest and operable in reconstituting a reporter enzyme activity upon association with the first reporter fragment pair member through the affinities of the interactor domains of the first and second reporter fragment pair members with the analyte of interest; and
(d) providing assay conditions in vitro sufficient to allow the first and second reporter fragment pair members to associate through the affinity of the interactor domains with the analyte, wherein reconstitution of the reporter enzyme activity indicates the presence of the analyte in the sample.

23. The method of claim 22, wherein the analyte is a divalent cation.

24. The method of claim 22, wherein the analyte is an antibody.

25-26. (canceled)

27. A reporter system comprising a first component comprising a first polypeptide reporter subunit and a second component comprising a second polypeptide reporter subunit, the first subunit and second subunit being capable of associating to generate an active polypeptide complex having enzyme activity which is capable of generating a detectable signal, said association being mediated by binding of the first and second components to an analyte of interest; wherein the first polypeptide subunit and/or the second polypeptide reporter subunit comprise one or more amino acid sequence changes which reduce the susceptibility of the active polypeptide complex to inhibition of said enzyme activity by an inhibitor.

28. A reporter system according to claim 27 wherein the inhibitor is a substance present in a biological sample.

29. A reporter system according to claim 28 wherein the substance present in the sample is an antibiotic.

30. A reporter system according to claim 27 wherein the active polypeptide complex has beta-lactamase activity.

31. A reporter system according to claim 30 wherein the first polypeptide reporter subunit comprises an alpha fragment of a beta lactamase and the second polypeptide reporter subunit comprises an omega fragment of a beta lactamase.

32. A reporter system according to claim 31 wherein the beta lactamase is TEM-1 beta lactamase and said amino acid sequence changes in the first polypeptide subunit comprise a M69L or M691 substitution.

33. A reporter system according to claim 31 wherein the beta lactamase is TEM-1 beta lactamase and said amino acid sequence changes in the second polypeptide subunit comprise a N276D substitution.

34. A reporter system according to claim 27 wherein the first and/or second reporter polypeptide subunits comprise one or more amino acid changes that enhance the stability and/or solubility of the subunits in vitro.

35. A reporter system according to claim 34 wherein the first polypeptide subunit comprises an alpha fragment of a TEM-1 beta lactamase and the second polypeptide subunit comprises an omega fragment of a TEM-1 beta lactamase and said amino acid sequence changes in the first polypeptide subunit comprise a V74T substitution and/or an M182T substitution.

36. A reporter system according to claim 34 wherein the first polypeptide subunit comprises an alpha fragment of a TEM-1 beta lactamase and the second polypeptide subunit comprises an omega fragment of a TEM-1 beta lactamase and said amino acid sequence changes in the second polypeptide subunit comprise an M211Q substitution.

37. A reporter system according to claim 27 wherein the first and second polypeptide subunits each comprise a flexible peptide linker which links the subunits to a first and second interactor domain, respectively.

38. A reporter system according to claim 27 wherein the first and second components are in substantially isolated and purified form.

39. A reporter system according to claim 27 wherein the first and/or second polypeptide subunits have been produced recombinantly in a bacterial cell, extracted from the cell under denaturing conditions, bound to a solid matrix and then refolded into an active conformation whilst bound to the solid matrix.

40. (canceled)

41. A method of determining the presence of an analyte of interest in a sample which method comprises contacting the sample with a reporter system according to claim 27 and detecting the presence or absence of enzyme activity resulting from the association of the first and second polypeptide subunits.

42-43. (canceled)

44. A method according to claim 41 wherein the analyte is an antibody which binds to a viral or bacterial antigen.

45. A method according to claim 41 wherein the analyte is a viral or bacterial antigen.

46. (canceled)


SEQUENCE LISTING (85 sequences)

SEQ ID NO: 1 - DNA, 552 bp, Artificial Sequence
  PB11 construct
atgcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcggtggaggtggctcgcatcaccatcatcatcactga

SEQ ID NO: 2 - protein, 183 aa, Artificial Sequence
  PB11 construct
MHPETLVKVKDAEDQLGARVGYIELDLNSGKILESFRPEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGGGGGSHHHHHH

SEQ ID NO: 3 - DNA, 315 bp, Artificial Sequence
  PB13 construct
atgcatcaccatcatcatcacggtggaggtggctcggaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtga

SEQ ID NO: 4 - protein, 104 aa, Artificial Sequence
  PB13 construct
MHHHHHHGGGGSELLTLASRQQLIDWMEADKVAGPLLRSALPAGWFIADKSGAGERGSRGIIAALGPDGKPSRIVVIYTTGSQATMDERNRQIAEIGASLIKHW

SEQ ID NO: 5 - DNA, 552 bp, Artificial Sequence
  PB11.2 construct
atgcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaaacgctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgacacctgtagcaatggcaacaacgttgcgcaaactattaactggcggtggaggtggctcgcatcaccatcatcatcactga

SEQ ID NO: 6 - protein, 183 aa, Artificial Sequence
  PB11.2 construct
MHPETLVKVKDAEDQLGARVGYIELDLNSGKILESFRPEERFPMMSTFKTLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTTPVAMATTLRKLLTGGGGGSHHHHHH

SEQ ID NO: 7 - DNA, 552 bp, Artificial Sequence
  PB11.11 construct (aM69L fragment)
atgcacccagaaacgctggtgaaagtaaaagatgctgaagatcagttgggtgcacgagtgggttacatcgaactggatctcaacagcggtaagatccttgagagttttcgccccgaagaacgttttccaatgctgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcggtggaggtggctcgcatcaccatcatcatcactga

SEQ ID NO: 8 - protein, 183 aa, Artificial Sequence
  PB11.11 (aM69L fragment)
MHPETLVKVKDAEDQLGARVGYIELDLNSGKILESFRPEERFPMLSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGGGGGSHHHHHH

SEQ ID NO: 9 - DNA, 552 bp, Artificial Sequence
  PB11.12 construct (wM69LM182T)
atgcacccagaaacgctggtgaaagtaaaagatgctgaagatcagttgggtgcacgagtgggttacatcgaactggatctcaacagcggtaagatccttgagagttttcgccccgaagaacgttttccaatgctgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgacacctgtagcaatggcaacaacgttgcgcaaactattaactggcggtggaggtggctcgcatcaccatcatcatcactga

SEQ ID NO: 10 - protein, 183 aa, Artificial Sequence
  PB11.12 construct (wM69LM182T)
MHPETLVKVKDAEDQLGARVGYIELDLNSGKILESFRPEERFPMLSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTTPVAMATTLRKLLTGGGGGSHHHHHH

SEQ ID NO: 11 - DNA, 315 bp, Artificial Sequence
  PB13.1 construct (M211Q substitution)
atgcatcaccatcatcatcacggtggaggtggctcggaactacttactctagcttcccggcaacaattaatagactggcaggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtga

SEQ ID NO: 12 - protein, 104 aa, Artificial Sequence
  PB13.1 construct (M211Q substitution)
MHHHHHHGGGGSELLTLASRQQLIDWQEADKVAGPLLRSALPAGWFIADKSGAGERGSRGIIAALGPDGKPSRIVVIYTTGSQATMDERNRQIAEIGASLIKHW

SEQ ID NO: 13 - DNA, 552 bp, Artificial Sequence
  PB11.13 construct (wM69IM182T fragment)
atgcacccagaaacgctggtgaaagtaaaagatgctgaagatcagttgggtgcacgagtgggttacatcgaactggatctcaacagcggtaagatccttgagagttttcgccccgaagaacgttttccaatgattagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgacacctgtagcaatggcaacaacgttgcgcaaactattaactggcggtggaggtggctcgcatcaccatcatcatcactga

SEQ ID NO: 14 - protein, 183 aa, Artificial Sequence
  PB11.13 construct (wM69IM182T fragment)
MHPETLVKVKDAEDQLGARVGYIELDLNSGKILESFRPEERFPMISTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTTPVAMATTLRKLLTGGGGGSHHHHHH

SEQ ID NO: 15 - DNA, 582 bp, Artificial Sequence
  PB11.4 construct
atgcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcggtggaggtggaagcggcggaggtggttctggtggtggaggctcgcatcaccatcatcatcactga

SEQ ID NO: 16 - protein, 193 aa, Artificial Sequence
  PB11.4 construct
MHPETLVKVKDAEDQLGARVGYIELDLNSGKILESFRPEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGGGGGSGGGGSGGGGSHHHHHH

SEQ ID NO: 17 - DNA, 345 bp, Artificial Sequence
  PB13.2 construct
atgcatcaccatcatcatcacggtggtggaggcagcggcggaggtggttctggtggaggtggctcggaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtga

SEQ ID NO: 18 - protein, 114 aa, Artificial Sequence
  PB13.2 construct
MHHHHHHGGGGSGGGGSGGGGSELLTLASRQQLIDWMEADKVAGPLLRSALPAGWFIADKSGAGERGSRGIIAALGPDGKPSRIVVIYTTGSQATMDERNRQIAEIGASLIKHW

SEQ ID NO: 19 - DNA, 315 bp, Artificial Sequence
  PB13.3 construct (wN276D fragment)
atgcatcaccatcatcatcacggtggaggtggctcggaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgagatagacagatcgctgagataggtgcctcactgattaagcattggtga

SEQ ID NO: 20 - protein, 104 aa, Artificial Sequence
  PB13.3 construct (wN276D fragment)
MHHHHHHGGGGSELLTLASRQQLIDWMEADKVAGPLLRSALPAGWFIADKSGAGERGSRGIIAALGPDGKPSRIVVIYTTGSQATMDERDRQIAEIGASLIKHW

SEQ ID NO: 21 - protein, 277 aa, Artificial Sequence
  PB15
MHMHHHHHHEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHWGSGSGHPETLVKVKDAEDQLGARVGYIELDLNSGKILESFRH

SEQ ID NO: 22 - protein, 277 aa, Artificial Sequence
  PB15.3 (M182T, I208T, F230Y)
MHMHHHHHHEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTTPVAMATTLRKLLTGELLTLASRQQLTDWMEADKVAGPLLRSALPAGWYIADKSGAGERGSRGIIAALGPDGKPSRIVVIYTTGSQATMDERNRQIAEIGASLIKHWGSGSGHPETLVKVKDAEDQLGARVGYIELDLNSGKILESFRH

SEQ ID NO: 23 - protein, 161 aa, Artificial Sequence
  PB7.2
MHMHHHHHHEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTTPVAMATTLRKLLTGELLTLASRQQLTDWMEAD

SEQ ID NO: 24 - protein, 129 aa, Artificial Sequence
  PB9.1
MKVAGPLLRSALPAGWYIADKSGAGERGSRGIIAALGPDGKPSRIVVIYTTGSQATMDERNRQIAEIGASLIKHWGSGSGHPETLVKVKDAEDQLGARVGYIELDLNSGKILESFRHGGGGGSHHHHHH

SEQ ID NO: 25 - DNA, 19 bp, Artificial Sequence
  FEC10 primer
gctagttattgctcagcgg

SEQ ID NO: 26 - DNA, 21 bp, Artificial Sequence
  FEC11 primer
taatacgactcactatagggg

SEQ ID NO: 27 - DNA, 40 bp, Artificial Sequence
  FEC16 primer
cttttccctgtccatatgcacccagaaacgctggtgaaag

SEQ ID NO: 28 - DNA, 79 bp, Artificial Sequence
  FEC24 primer
cttttccctgtccatatgcatcaccatcatcatcacggtggaggtggctcggaactacttactctagcttcccggcaac

SEQ ID NO: 29 - DNA, 84 bp, Artificial Sequence
  FEC27 primer
aaccggtattccctcgagtcagtgatgatgatggtgatgcgagccacctccaccgccagttaatagtttgcgcaacgttgttgc

SEQ ID NO: 30 - DNA, 45 bp, Artificial Sequence
  FEC29 primer
aaccggtattccctcgagtcaccaatgcttaatcagtgaggcacc

SEQ ID NO: 31 - DNA, 26 bp, Artificial Sequence
  FEC47 primer
gcacttttaaaacgctgctatgtggc

SEQ ID NO: 32 - DNA, 26 bp, Artificial Sequence
  FEC48 primer
gccacatagcagcgttttaaaagtgc

SEQ ID NO: 33 - DNA, 31 bp, Artificial Sequence
  FEC49 primer
caattaatagactggcaggaggcggataaag

SEQ ID NO: 34 - DNA, 31 bp, Artificial Sequence
  FEC50 primer
ctttatccgcctcctgccagtctattaattg

SEQ ID NO: 35 - DNA, 31 bp, Artificial Sequence
  FEC55 primer
gtgacaccacgacacctgtagcaatggcaac

SEQ ID NO: 36 - DNA, 31 bp, Artificial Sequence
  FEC56 primer
gttgccattgctacaggtgtcgtggtgtcac

SEQ ID NO: 37 - DNA, 51 bp, Artificial Sequence
  FEC67 primer
ctattaactggcggtggaggtggaagcggcggaggtggttctggtggtgga

SEQ ID NO: 38 - DNA, 58 bp, Artificial Sequence
  FEC68 primer
gtcagtgatgatgatggtgatgcgagcctccaccaccagaaccacctccgccgcttcc

SEQ ID NO: 39 - DNA, 52 bp, Artificial Sequence
  FEC75 primer
ggtggtggaggcagcggcggaggtggttctggtggaggtggctcggaactac

SEQ ID NO: 40 - DNA, 54 bp, Artificial Sequence
  FEC76 primer
agaaccacctccgccgctgcctccaccaccgtgatgatgatggtgatgcatatg

SEQ ID NO: 41 - DNA, 40 bp, Artificial Sequence; identical to SEQ ID NO: 27
  PAN1 primer

SEQ ID NO: 42 - DNA, 42 bp, Artificial Sequence
  PAN2 primer
ggaataccggttctcgagccaatgcttaatcagtgaggcacc

SEQ ID NO: 43 - DNA, 84 bp, Artificial Sequence; identical to SEQ ID NO: 29
  PAN3 primer

SEQ ID NO: 44 - DNA, 79 bp, Artificial Sequence; identical to SEQ ID NO: 28
  PAN4 primer

SEQ ID NO: 45 - DNA, 45 bp, Artificial Sequence; identical to SEQ ID NO: 30
  PAN5 primer

SEQ ID NO: 46 - DNA, 58 bp, Artificial Sequence
  PAN6 primer
ggaagcggcggaggtggttctggtggtggaggctcgcatcaccatcatcatcactgac

SEQ ID NO: 47 - DNA, 51 bp, Artificial Sequence
  PAN7 primer
tccaccaccagaaccacctccgccgcttccacctccaccgccagttaatag

SEQ ID NO: 48 - DNA, 52 bp, Artificial Sequence; identical to SEQ ID NO: 39
  PAN8 primer

SEQ ID NO: 49 - DNA, 54 bp, Artificial Sequence; identical to SEQ ID NO: 40
  PAN9 primer

SEQ ID NO: 50 - DNA, 45 bp, Artificial Sequence
  PAN10 primer
cctctcgagtcagtgatgatgatgatggtgactaccgccaccgcc

SEQ ID NO: 51 - DNA, 85 bp, Artificial Sequence
  PAN12 primer
cggaggtggttctggtggtggaggctcgcatcttgaaggcggcgatggcacaagagatacccttcctcagagtccaggtccggcg

SEQ ID NO: 52 - DNA, 85 bp, Artificial Sequence
  PAN12 primer
ggatctgggctcggcactactggtctgttcggtttatctttctccacatcttccgccagtggaaacgccggacctggactctgag

SEQ ID NO: 53 - DNA, 85 bp, Artificial Sequence
  PAN13 primer
gtagtgccgagcccagatccgaacaacagcccagcgagaccggaaaccagcagaccgaaaacccctcccggcggtggcggtagtc

SEQ ID NO: 54 - DNA, 46 bp, Artificial Sequence
  PAN14 primer
cgccatatgcacccagaaacgctggtgaaagtaaaagatgctgaag

SEQ ID NO: 55 - DNA, 77 bp, Artificial Sequence
  PAN15 primer
ctggtggtggaggctcggctcctcctcctcctgaacatcgtggcggcccggaagaatttgaaggcgctggcgatggc

SEQ ID NO: 56 - DNA, 75 bp, Artificial Sequence
  PAN16 primer
cgggttcggggtacgaaacgccagaccagttgcactatcatcatcttccggcggttcgccatcgccagcgccttc

SEQ ID NO: 57 - DNA, 77 bp, Artificial Sequence
  PAN17 primer
gtttcgtaccccgaacccgaacaaaccgccgcctgcacgtcctggtcctattcgtccgaccctgccgcctggcattc

SEQ ID NO: 58 - DNA, 74 bp, Artificial Sequence
  PAN18 primer
ctttcgccggagcttgtgcaggaggacgaggggtgttcggagccagcggacctagaatgccaggcggcagggtc

SEQ ID NO: 59 - DNA, 81 bp, Artificial Sequence
  PAN19 primer
gcacaagctccggcgaaagatatgccgagtggcccgaccccgcagcatattcctctgttttggggcggtggcggtagtcac

SEQ ID NO: 60 - DNA, 41 bp, Artificial Sequence
  PAN20 primer
gtttaacttcgcagatctcatctggaaggcggcgatggcac

SEQ ID NO: 61 - DNA, 36 bp, Artificial Sequence
  PAN21 primer
ccactagtcggcggggttttcgggcggctggtttcc

SEQ ID NO: 62 - DNA, 31 bp, Artificial Sequence
  PAN22 primer
cgcggatccgcgccgccgccgccggaacatc

SEQ ID NO: 63 - DNA, 45 bp, Artificial Sequence
  PAN23 primer
ccactagtccaaaacagcggaatatgctgcggggtcgggccgctc

SEQ ID NO: 64 - DNA, 30 bp, Artificial Sequence
  PAN24 primer
atggatgaacgagatagacagatcgctgag

SEQ ID NO: 65 - DNA, 30 bp, Artificial Sequence
  PAN25 primer
ctcagcgatctgtctatctcgttcatccat

SEQ ID NO: 66 - DNA, 171 bp, Artificial Sequence
  HSV-1 truncated antigen displaying sequence of P1-1 trunc.
catcttgaaggcggcgatggcacaagagatacccttcctcagagtccaggtccggcgtttccactggcggaagatgtggagaaagataaaccgaacagaccagtagtgccgagcccagatccgaacaacagcccagcgagaccggaaaccagcagaccgaaaacccctccc

SEQ ID NO: 67 - protein, 57 aa, Artificial Sequence
  HSV-1 truncated antigen displaying sequence of P1-1 trunc.
HLEGGDGTRDTLPQSPGPAFPLAEDVEKDKPNRPVVPSPDPNNSPARPETSRPKTPP

SEQ ID NO: 68 - DNA, 768 bp, Artificial Sequence
  HSV-1 alpha reporter fragment displaying sequence of BLaHSV1 construct
atgcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcggtggaggtggaagcggcggaggtggttctggtggtggaggctcgcatcttgaaggcggcgatggcacaagagatacccttcctcagagtccaggtccggcgtttccactggcggaagatgtggagaaagataaaccgaacagaccagtagtgccgagcccagatccgaacaacagcccagcgagaccggaaaccagcagaccgaaaacccctcccggcggtggcggtagtcaccatcatcatcatcactga

SEQ ID NO: 69 - protein, 255 aa, Artificial Sequence
  HSV-1 alpha reporter fragment displaying sequence of BLaHSV1 construct
MHPETLVKVKDAEDQLGARVGYIELDLNSGKILESFRPEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGGGGGSGGGGSGGGGSHLEGGDGTRDTLPQSPGPAFPLAEDVEKDKPNRPVVPSPDPNNSPARPETSRPKTPPGGGGSHHHHHH

SEQ ID NO: 70 - DNA, 549 bp, Artificial Sequence
  HSV-1 omega reporter fragment displaying sequence of BLwHSV1 construct
atgcatcaccatcatcatcacggtggtggaggcagcggatctcatcttgaaggcggcgatggcacaagagatacccttcctcagagtccaggtccggcgtttccactggcggaagatgtggagaaagataaaccgaacagaccagtagtgccgagcccagatccgaacaacagcccagcgagaccggaaaccagcagaccgaaaacccctcccactagtggtggtggaggcagcggcggaggtggttctggtggaggtggctcggctagcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgagatagacagatcgctgagataggtgcctcactgattaagcattggtaa

SEQ ID NO: 71 - protein, 182 aa, Artificial Sequence
  HSV-1 omega reporter fragment displaying sequence of BLwHSV1 construct
MHHHHHHGGGGSGSHLEGGDGTRDTLPQSPGPAFPLAEDVEKDKPNRPVVPSPDPNNSPARPETSRPKTPPTSGGGGSGGGGSGGGGSASELLTLASRQQLIDWMEADKVAGPLLRSALPAGWFIADKSGAGERGSRGIIAALGPDGKPSRIVVIYTTGSQATMDERDRQIAEIGASLIKHW

SEQ ID NO: 72 - DNA, 273 bp, Artificial Sequence
  HSV-2 truncated antigen displaying sequence of P2-1 trunc.
gctcctcctcctcctgaacatcgtggcggcccggaagaatttgaaggcgctggcgatggcgaaccgccggaagatgatgatagtgcaactggtctggcgtttcgtaccccgaacccgaacaaaccgccgcctgcacgtcctggtcctattcgtccgaccctgccgcctggcattctaggtccgctggctccgaacacccctcgtcctcctgcacaagctccggcgaaagatatgccgagtggcccgaccccgcagcatattcctctgttttgg

SEQ ID NO: 73 - protein, 91 aa, Artificial Sequence
  HSV-2 truncated antigen displaying sequence of P2-1 trunc.
APPPPEHRGGPEEFEGAGDGEPPEDDDSATGLAFRTPNPNKPPPARPGPIRPTLPPGILGPLAPNTPRPPAQAPAKDMPSGPTPQHIPLFW

SEQ ID NO: 74 - DNA, 870 bp, Artificial Sequence
  HSV-2 alpha reporter fragment displaying sequence of BLaHSV2 construct
atgcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcggtggaggtggaagcggcggaggtggttctggtggtggaggctcggctcctcctcctcctgaacatcgtggcggcccggaagaatttgaaggcgctggcgatggcgaaccgccggaagatgatgatagtgcaactggtctggcgtttcgtaccccgaacccgaacaaaccgccgcctgcacgtcctggtcctattcgtccgaccctgccgcctggcattctaggtccgctggctccgaacacccctcgtcctcctgcacaagctccggcgaaagatatgccgagtggcccgaccccgcagcatattcctctgttttggggcggtggcggtagtcaccatcatcatcatcactga

SEQ ID NO: 75 - protein, 289 aa, Artificial Sequence
  HSV-2 alpha reporter fragment displaying sequence of BLaHSV2 construct
MHPETLVKVKDAEDQLGARVGYIELDLNSGKILESFRPEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGGGGGSGGGGSGGGGSAPPPPEHRGGPEEFEGAGDGEPPEDDDSATGLAFRTPNPNKPPPARPGPIRPTLPPGILGPLAPNTPRPPAQAPAKDMPSGPTPQHIPLFWGGGGSHHHHHH

SEQ ID NO: 76 - DNA, 651 bp, Artificial Sequence
  HSV-2 omega reporter fragment displaying sequence of BLwHSV2 construct
atgcatcaccatcatcatcacggtggtggaggcagcggatccgctcctcctcctcctgaacatcgtggcggcccggaagaatttgaaggcgctggcgatggcgaaccgccggaagatgatgatagtgcaactggtctggcgtttcgtaccccgaacccgaacaaaccgccgcctgcacgtcctggtcctattcgtccgaccctgccgcctggcattctaggtccgctggctccgaacacccctcgtcctcctgcacaagctccggcgaaagatatgccgagtggcccgaccccgcagcatattcctctgttttggactagtggtggtggaggcagcggcggaggtggttctggtggaggtggctcggctagcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgagatagacagatcgctgagataggtgcctcactgattaagcattggtaa

SEQ ID NO: 77 - protein, 216 aa, Artificial Sequence
  HSV-2 omega reporter fragment displaying sequence of BLwHSV2 construct
MHHHHHHGGGGSGSAPPPPEHRGGPEEFEGAGDGEPPEDDDSATGLAFRTPNPNKPPPARPGPIRPTLPPGILGPLAPNTPRPPAQAPAKDMPSGPTPQHIPLFWTSGGGGSGGGGSGGGGSASELLTLASRQQLIDWMEADKVAGPLLRSALPAGWFIADKSGAGERGSRGIIAALGPDGKPSRIVVIYTTGSQATMDERDRQIAEIGASLIKHW

SEQ ID NO: 78 - DNA, 165 bp, Artificial Sequence
  Protein G subunit
acctataaactggtgattaacggcaaaaccctgaaaggcgaaaccaccaccgaagcggtggatgcggcgaccgcggaaaaagtgtttaaacagtatgcgaacgataacggcgtggatggcgaatggacctatgatgatgcgactaagacctttacggtaactgaa

SEQ ID NO: 79 - protein, 55 aa, Artificial Sequence
  Protein G subunit
TYKLVINGKTLKGETTTEAVDAATAEKVFKQYANDNGVDGEWTYDDATKTFTVTE

SEQ ID NO: 80 - DNA, 780 bp, Artificial Sequence
  Protein G subunit alpha reporter fragment displaying sequence of BLaProG construct
atgcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcggtaccggtggtggaggcagcggcggaggtggttctggtggaggtggctcgggatccacctataaactggtgattaacggcaaaaccctgaaaggcgaaaccaccaccgaagcggtggatgcggcgaccgcggaaaaagtgtttaaacagtatgcgaacgataacggcgtggatggcgaatggacctatgatgatgcgactaagacctttacggtaactgaaactagtggcggcggcggcagccatcatcatcatcatcattaa

SEQ ID NO: 81 - protein, 259 aa, Artificial Sequence
  Protein G subunit alpha reporter fragment displaying sequence of BLaProG construct
MHPETLVKVKDAEDQLGARVGYIELDLNSGKILESFRPEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGGTGGGGSGGGGSGGGGSGSTYKLVINGKTLKGETTTEAVDAATAEKVFKQYANDNGVDGEWTYDDATKTFTVTETSGGGGSHHHHHH

SEQ ID NO: 82 - DNA, 543 bp, Artificial Sequence
  Protein G subunit omega reporter fragment displaying sequence of BLaProGconstruct
atgcatcaccatcatcatcacggtggtggaggcagcggatccacctataaactggtgattaacggcaaaaccctgaaaggcgaaaccaccaccgaagcggtggatgcggcgaccgcggaaaaagtgtttaaacagtatgcgaacgataacggcgtggatggcgaatggacctatgatgatgcgactaagacctttacggtaactgaaactagtggtggtggaggcagcggcggaggtggttctggtggaggtggctcggctagcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgagatagacagatcgctgagataggtgcctcactgattaagcattggtaa

SEQ ID NO: 83 - protein, 180 aa, Artificial Sequence
  Protein G subunit omega reporter fragment displaying sequence of BLaProGconstruct
MHHHHHHGGGGSGSTYKLVINGKTLKGETTTEAVDAATAEKVFKQYANDNGVDGEWTYDDATKTFTVTETSGGGGSGGGGSGGGGSASELLTLASRQQLIDWMEADKVAGPLLRSALPAGWFIADKSGAGERGSRGIIAALGPDGKPSRIVVIYTTGSQATMDERDRQIAEIGASLIKHW

SEQ ID NO: 84 - protein, 57 aa, Artificial Sequence; identical to SEQ ID NO: 67
  Amino acids 92 to 148 of gG1 of HSV1

SEQ ID NO: 85 - protein, 91 aa, Artificial Sequence; identical to SEQ ID NO: 73
  Amino acids 551 to 641 of gG2 of HVS2